(19) United States
(12) Patent Application Publication
(10) Pub. No.: US 2024/0327920 A1
(43) Pub. Date: Oct. 3, 2024

(54) Small Deletion Signatures

(71) Applicant: The Jackson Laboratory, Bar Harbor, ME (US)

(72) Inventors: Roel Verhaak, Farmington, CT (US); Kevin Anderson, Farmington, CT (US); Emre Kocakavuk, Farmington, CT (US); Floris Barthel, Farmington, CT (US)

(21) Appl. No.: 18/001,841

(22) PCT Filed: May 26, 2021
(86) PCT No.: PCT/US2021/034297

§ 371 Date: Jul. 24, 2023

Related U.S. Application Data
(60) Provisional application No. 63/038,847, filed on Jun. 14, 2020.

(51) Int. Cl. C12Q 1/6886

(52) U.S. Cl. CPC C12Q 1/6886; C12Q 1/6869; C12Q 2600/106; C12Q 2600/156

(57) ABSTRACT

The invention relates, in part, to methods of assessing small deletion signatures to determine treatments for cancers and to determine a prognosis for a subject having a cancer.

### RELATED APPLICATIONS

This application claims the benefit under 35 U.S.C. § 119(e) of U.S. Provisional application Ser. No. 63/038,847, filed Jun. 14, 2020, the disclosure of which is incorporated by reference herein in its entirety.

### GOVERNMENT SUPPORT

This invention was made with government support under NIH/NCI R01 CA190121, R01 CA237208, NIH/NINDS R21 NS 114873 awarded by the National Institutes of Health and W81XWH1910246 awarded by the Department of Defense. The government has certain rights in the invention.

### FIELD OF THE INVENTION

The invention relates, in part, to methods of assessing small deletion signatures to determine treatments for cancers and to determine a prognosis for a subject having a cancer.

### BACKGROUND OF THE INVENTION

Radiation therapy or radiotherapy is a main stay of cancer therapy, used in clinical management of more than half of cancer patients. While conferring both survival and palliative benefits, side effects include cognitive dysfunction and cardiomyopathies. As cancer is an evolutionary disease, the identification of therapy-associated mutations implicates both that the therapy had an effect on the tumor, and that a therapy-resistant tumor subclone has emerged. A well-known example of such a process is the hypermutation following temozolomide therapy which is observed in a significant proportion of gliomas [Touat, M. et al., Nature 580(7804), 517-523 (2020); Barthel, F. P. et al., Nature 576, 112-120 (2019)]. Similarly, an increased burden of small deletions has been observed in radiation-induced malignancies [Behjati, S. et al., Nat. Commun., 7, 12605 (2016)]. Despite these advancements, the mutational footprints of therapeutic radiation are not well understood.

### SUMMARY OF THE INVENTION

According to an aspect of the invention, a method of selecting a cancer treatment for a subject is provided, the method including: (a) obtaining a biological sample from a subject that has or is believed at risk for having a cancer; (b) determining at least one characteristic of a plurality of small deletions in somatic DNA in the biological sample; (c) comparing the determined small deletions characteristic to a control of the small deletion characteristic; and (d) selecting a treatment for the subject based at least in part on the comparison of the determined characteristic of the small deletions with the control. In some embodiments, the treatment selected includes abstention from administering a radiotherapy to the subject. In some embodiments, the treatment selected includes administering a radiotherapy to the subject. In some embodiments, the at least one characteristic is one or more of: presence of the plurality of small deletions, small deletion burden, absence of the plurality of small deletions, absolute quantity of the small deletions in the plurality of small deletions, relative quantity of the small deletions in the plurality of small deletions, size of the small deletions in the plurality of small deletions, and genetic identity of one or more of the plurality of the small deletions. In some embodiments, a means for determining the at least one characteristic of the plurality of small deletions includes one or more of exome sequencing and whole genome sequencing. In some embodiments, the subject has been administered a radiotherapy before the biological sample is obtained. In some embodiments, the radiotherapy was administered to treat a cancer in the subject. In some embodiments, the subject has a metastatic cancer. In some embodiments, the subject has a recurrence of a previous cancer. In some embodiments, the control of the one or more small deletion characteristics includes the one or more small deletion characteristics previously determined for the subject. In some embodiments, the characteristic of the plurality of small deletions is a number of the small deletions and if: (a) the number of small deletions determined in the subject sample is higher than a control number of small deletions, the selected treatment comprises abstaining from administering a radiotherapy to the subject, and (b) the number of small deletions determined in the subject sample is statistically significantly equal to or lower than a control number of small deletions, the selected treatment includes administering a radiotherapy to the subject. In some embodiments, the determined number of small deletions in the subject sample is at least 10%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500% higher than the control number of small deletions. In some embodiments, the determined number of small deletions in the subject sample is at least 0.25, 0.5, 1, 2, 3, 4, 5, 6, 7, 8, 9, 10-fold higher than the control number of small deletions. In some embodiments, if the control number of small deletions is zero and the determination of a number of small deletions in the subject sample is statistically significantly greater than zero, the selected treatment includes abstaining from administering a radiotherapy to the subject. In some embodiments, the characteristic of the plurality of small deletions is small deletion burden and if: (a) the small deletion burden determined in the subject sample is statistically significantly higher than a control small deletion burden, the selected treatment includes abstaining from administering a radiotherapy to the subject, and (b) the small deletion burden determined in the subject sample is statistically significantly lower or is equal to a control small deletion burden, the selected treatment includes administering a radiotherapy to the subject. In some embodiments, a means for determining the small deletion burden includes determining the at least one characteristic of the plurality of small deletions in the somatic DNA in the biological sample. In some embodiments, the determined small deletion burden in the subject sample is at least 10%, 20%, 25%, 30%, 35%, 40%, 45%, 50%, 55%, 60%, 65%, 70%7, 75%, 80%5, 85%, 90%, 95%, 100%, 150%, 200%, 250%, 300%, 350%, 400%, 450%, 500% higher than the control small deletion burden. In some embodiments, the determined small deletion burden in the subject sample is at least 0.25, 0.5, 1, 2, 3, 4, 5, 6, 7, 8, 9, 10-fold higher than the control small deletion burden. In some embodiments, if the control small deletion burden is zero and the determination of a small deletion burden is statistically significantly greater than zero in the subject sample, the selected treatment includes abstaining from administering a radiotherapy to the subject. In some embodiments, the selected treatment includes administering a palliative or curative radiotherapy to the subject. In some embodiments, the determining of the characteristic of the plurality of small deletions includes determining the identity of one or more specific small deletions in the plurality of small deletions. In some embodiments, a selected treatment also includes one or more of: surgery, chemotherapy, administration of a pharmaceutical agent, an immunotherapy, a modified T cell therapy, a virus-based therapy, abstention from surgery, abstention from chemotherapy, and abstention from administration of a pharmaceutical agent. In some embodiments, the biological sample includes one or more of: tissue, blood, serum, saliva, lymph fluid, and cerebrospinal fluid (CSF). In some embodiments, the biological sample includes circulating tumor DNA (ctDNA). In some embodiments, the biological sample includes cells of the subject. In some embodiments, the biological sample includes cancer cells of the subject. In some embodiments, the cancer is: a brain cancer, a bone cancer, a soft-tissue cancer, a breast cancer, a colon cancer, a rectal cancer, an esophageal cancer, a head and neck cancer, a lung cancer, an ovarian cancer, a prostate cancer, a skin cancer, a urinary tract cancer, or a uterine cancer. In some embodiments, the brain cancer is a glioma. In some embodiments, the subject is a mammal, optionally a human. In some embodiments, the method also includes treating the subject with one or more of the selected treatments. In some embodiments, the at least one characteristic of the plurality of small deletions is an amount of the small deletions and the amount is at least 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more small deletions per megabase of sequence. In some embodiments, the at least one characteristic of the plurality of small deletions is length of the small deletions in the plurality of small deletions and the small deletions are between 5 and 100 base pairs (bp) in length. In some embodiments, the at least one characteristic of the plurality of small deletions is length of the small deletions in the plurality of small deletions and the small deletions are between 5 and 50 bp in length. In some embodiments, the at least one characteristic of the plurality of small deletions is length of the small deletions in the plurality of small deletions and the small deletions are between 5 and 15 bp in length. In some embodiments, the at least one characteristic of the plurality of small deletions is length of the small deletions in the plurality of small deletions and the small deletions are less than 100 bp in length. In some embodiments, the at least one characteristic of the plurality of small deletions is length of the small deletions in the plurality of small deletions and the small deletions are of two or more different lengths. In some embodiments, the radiotherapy administered before the biological sample was obtained included a curative radiotherapy. In some embodiments, the radiotherapy administered before the biological sample was obtained included a palliative radiotherapy.

According to another aspect of the invention, a method of determining for a subject one or more of: an increased risk of a cancer and an increased mortality risk from a cancer is provided, the method including: (a) determining at least one characteristic of a plurality of small deletions in somatic DNA in a biological sample obtained from a subject that has been administered a radiotherapy; (b) comparing the determined small deletions characteristic to a control of the small deletion characteristic; and (c) determining the presence or absence of one or both of: an increased risk of a cancer in the subject and an increased mortality risk for a cancer in the subject based at least in part on the comparison of the determined characteristic of the small deletions with the control. In some embodiments, the at least one characteristic is: presence of the plurality of small deletions, small deletion burden, absence of the plurality of small deletions, a relative amount or quantity of the small deletions in the plurality of small deletions, an absolute amount or quantity of the small deletions in the plurality of small deletions; a length of small deletions in the plurality of small deletions, and genetic identity of one or more of the plurality of the small deletions. In some embodiments, a means for determining the at least one characteristic of a plurality of small deletions includes one or more of exome sequencing and whole genome sequencing. In some embodiments, the control of the small deletion characteristic includes a small deletion characteristic previously determined for the subject. In some embodiments, the control is a determination of the at least one characteristic of a plurality of small deletions in a sample obtained from the subject prior to the administered radiotherapy. In some embodiments, the administered radiotherapy was a palliative radiotherapy administered to treat a cancer in the subject. In some embodiments, the administered radiotherapy was a curative radiotherapy administered to treat a cancer in the subject. In some embodiments, the subject has metastatic cancer. In some embodiments, the subject has a recurrence of a previous cancer. In some embodiments, the characteristic of the plurality of small deletions is a number of the small deletions and if the number of small deletions determined in the subject sample is statistically significantly higher than a control number of small deletions, the prognosis is one or more of: an increased risk of a cancer in the subject and an increased mortality risk in the subject. In some embodiments, the determined number of small deletions in the subject sample is at least 10%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500% higher than the control number of small deletions. In some embodiments, the determined number of small deletions in the subject sample is at least 0.25, 0.5, 1, 2, 3, 4, 5, 6, 7, 8, 9, 10-fold higher than the control number of small deletions. In some embodiments, if the control number of small deletions is zero and the determination of a number of small deletions in the subject sample is statistically significantly greater than zero, the selected treatment includes abstaining from administering a radiotherapy to the subject. In some embodiments, the characteristic of the plurality of small deletions is small deletion burden and if. (a) the small deletion burden determined in the subject sample is higher than a control small deletion burden, the selected treatment includes abstaining from administering a radiotherapy to the subject, and (b) the small deletion burden determined in the subject sample is statistically significantly equal to or lower than a control small deletion burden, the selected treatment includes administering a radiotherapy to the subject. In some embodiments, a means for determining the small deletion burden includes determining the at least one characteristic of the plurality of small deletions in the somatic DNA in the biological sample. In some embodiments, the determined small deletion burden in the subject sample is at least 10%, 20%, 25%, 30%, 35%, 40%, 45%, 50%, 55%, 60%, 65%, 70%, 75%, 80%5, 85%, 90%, 95%, 100%, 150%, 200%, 250%, 300%, 350%, 400%, 450%, 500% higher than the control small deletion burden. In some embodiments, the determined small deletion burden in the subject sample is at least 0.25, 0.5, 1, 2, 3, 4, 5, 6, 7, 8, 9, 10-fold higher than the control small deletion burden. In some embodiments, if the control small deletion burden is zero and the determination of a small deletion burden is statistically significantly greater than zero in the subject sample, the selected treatment includes abstaining from administering a radiotherapy to the subject. In some embodiments, the determining of the characteristic of the plurality of small deletions includes determining the identity of one or more specific small deletions in the plurality of small deletions. In some embodiments, a selected treatment also includes one or more of: surgery, chemotherapy, administration of a pharmaceutical agent, an immunotherapy, a modified T cell therapy, a virus-based therapy, abstention from surgery, abstention from chemotherapy, and abstention from administration of a pharmaceutical agent. In some embodiments, the biological sample includes one or more of: tissue, blood, serum, saliva, lymph fluid, and cerebrospinal fluid (CSF). In some embodiments, the biological sample includes circulating tumor DNA (ctDNA). In some embodiments, if the presence of increased risk of the cancer in the subject is determined, the subject has at least 5%, 10%, 15%, 20%, 25%, 30%, 35%, 40%, 45%, 50%, 55%, 60%, 65%, 70%, 75%, 80%5, 85%, 90%, 95%, 100%, 150%, 200%, 250%, 300%, 350%, 400%, 450%, 500% greater risk for a cancer than if the absence of increased risk was determined for the subject. In some embodiments, if the presence of increased mortality from cancer is determined for the subject, the subject has at least 5%, 10%, 15%, 20%, 25%, 30%, 35%, 40%, 45%, 50%, 55%, 60%, 65%, 70%, 75%, 80%5, 85%, 90%, 95%, 100%, 150%, 200%, 250%, 300%, 350%, 400%, 450%, 500% greater risk of mortality from a cancer than if the absence of increased risk of mortality was determined for the subject. In some embodiments, the risk of mortality is determined as a function of time prior to death. In some embodiments, the time is one or more of days, weeks, months, and years. In some embodiments, the biological sample comprises cells of the subject. In some embodiments, the biological sample includes cancer cells of the subject. In some embodiments, the biological sample is a tissue sample. In some embodiments, the biological sample includes circulating tumor DNA (ctDNA). In some embodiments, the cancer is: a brain cancer, a bone cancer, a soft-tissue cancer, a breast cancer, a colon cancer, a rectal cancer, a central nervous system cancer, an esophageal cancer, a head and neck cancer, a lung cancer, an ovarian cancer, a prostate cancer, a skin cancer, a urinary tract cancer, or a uterine cancer. In some embodiments, the brain cancer is a glioma. In some embodiments, the subject is a mammal, optionally a human. In some embodiments, the method also includes treating the subject with one or more cancer treatments. In some embodiments, the one or more cancer treatments comprise one or more of: surgery, chemotherapy, administration of a pharmaceutical agent, abstention from surgery, abstention from chemotherapy, abstention from administration of a pharmaceutical agent, and abstention from radiotherapy. In some embodiments, the plurality of small deletions includes at least 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more small deletions per megabase of sequence. In some embodiments, the at least one characteristic of the plurality of small deletions is length of the small deletions in the plurality of small deletions and the small deletions are between 5 and 100 base pairs (bp) in length. In some embodiments, the at least one characteristic of the plurality of small deletions is length of the small deletions in the plurality of small deletions and the small deletions are between 5 and 50 bp in length. In some embodiments, the at least one characteristic of the plurality of small deletions is length of the small deletions in the plurality of small deletions and the small deletion are between 5 and 15 bp in length. In some embodiments, the at least one characteristic of the plurality of small deletions is length of the small deletions in the plurality of small deletions and the small deletions are less than 100 bp in length. In some embodiments, the at least one characteristic of the plurality of small deletions is length of the small deletions in the plurality of small deletions comprise small deletions of two or more different lengths.

### BRIEF DESCRIPTION OF THE DRAWINGS

FIG. 1A-D provides graphs illustrating that radiotherapy was associated with an increased small deletion burden. FIG. 1A shows a boxplot depicting the burden of newly acquired/post-treatment small deletions (deletions/Mb) in RT-naïve (RT−, n=34) and RT-received (RT+, n=156) patients from the GLASS cohort. Mann-Whitney U test was applied for statistical testing. FIG. 1B shows violin plots of longitudinal comparison of small deletion burden between primary and recurrent glioma samples, separated by hypermutation (HM) and Radiotherapy (RT). Paired Wilcoxon signed-rank test was applied for statistical testing. FIG. 1C shows a forest plot showing a multivariable log-linear regression model of newly acquired small deletion burden (deletions/Mb) including (1) temozolomide (TMZ)-treatment, (2) hypermutation (HM), (3) RT-treatment, (4) molecular subtype and (5) surgical interval (in months) as variables. OR, odds ratio; CI, confidence interval. FIG. 1D, upper row, shows boxplots depicting small deletion burden (deletions/Mb) in in metastatic cohort tumor samples separated by primary tumor location. For each individual panel of three boxplots, RT-naïve (RT−, left), RT-treated with palliative intent (RT+ pal, middle), and RT-treated with curative intent (RT+ cur, right). Statistical testing, Kruskal-Wallis test. FIG. 1D, lower row, shows bar graphs depicting sample sizes of the metastatic cohort separated by primary tumor location. Each bar graph panel corresponds to the boxplot panel immediately above.

FIG. 2A-J provides graphs and a flowchart illustrating the association of radiotherapy with an increased burden of newly acquired/post-treatment mutations. FIG. 2A shows boxplots comparing the burden of several types of newly acquired/post-treatment mutations (mutations/Mb) in RT-naïve (RT−, n=34) and RT-treated (RT+, n=156) patients from the GLASS cohort. Mutations were separated by small deletions (DEL), small insertions (INS), and single nucleotide variants (SNV). Statistical testing, Mann-Whitney U test. FIG. 2B shows boxplots comparing newly acquired small deletion burdens (mutations/Mb) between RT-naïve (RT−) and RT-treated (RT+) cases separated by molecular subtype (IDHmut vs. IDHwt). Statistical testing, Mann-Whitney U test. FIG. 2C shows boxplots comparing the mean cancer cell fractions of small deletions per patient in the GLASS cohort, separated by P (primary-only fraction, pretreatment), S (shared fraction, pre-treatment), and R (recurrence only fraction, post-treatment), and by HM (hypermutation) versus non-HM (non-hypermutation) status. Statistical testing, Mann-Whitney U test. FIG. 2D shows forest plots showing a multivariable log-linear regression model of newly acquired mutation burdens (mutations/mb) in the GLASS cohort for the following variables: TMZ-treatment, hypermutation, RT-treatment, molecular subtype, and surgical interval (in months). Mutation types were separated into small deletions (circle), small insertions (square), indels (diamond, small deletions+ small insertions), SNVs (triangle, single nucleotide variants), and overall tumor mutational burden (inverted triangle; TMB, small indels+SNVs). A point indicates a mean estimate of the model; lines indicate 95% confidence intervals. Hypermutation was significantly associated with increased burden of all types of mutations, and RT was associated with a slightly increased burden of small deletions and indels (potentially driven by the large effect size of small deletions). FIG. 2E shows a flowchart of sample selection and filtering criteria for the metastatic cohort. FIG. 2F shows boxplots comparing small deletion burdens between RT−, RT+ pal, and RT+ cur samples, respectively, for breast, lung, and bone/soft tissue cancers separated into their respective subtypes. Statistical testing, Kruskal-Wallis test. FIG. 2G shows boxplots depicting small deletion burdens in HRD−/MSI− (n=3,413), HRD+(n=218), and MSI+(n=62) samples from the HMF cohort separated by radiotherapy treatment status (homologous recombination deficiency (HRD), microsatellite instability (MSI)). Statistical testing, Mann-Whitney U test. FIG. 2H shows forest plots depicting a multivariable log-linear regression model for mutation burdens in the metastatic cohort. Mutations were separated into small deletions, small insertions, and SNVs. Independent variables included age, tumor type (primary tumor location), DNA repair deficiency background, and various treatment types including radiotherapy, taxane, alkylating agents, platin, and others. FIG. 2I shows boxplots comparing small deletion counts between control vs. ionizing radiation groups from a previously described dataset [Kucab et al., Cell 177, 821-836.e16. (2019)]. Statistical testing, Mann-Whitney U test. FIG. 2J shows a bar graph of the distribution of small deletion counts per treatment group from a previously described dataset [Kucab et al., Cell 177, 821-836.e16. (2019)]. Bars indicate means, error bars reflect standard deviation, and dots indicate the median count of small deletions. The ionizing radiation group displayed the highest median counts of small deletions. PAH, polycyclic aromatic hydrocarbon; ROS, reactive oxygen species; UV, ultraviolet; DDR, DNA damage response.

FIG. 3A-D presents graphs and a schematic illustrating distribution of small deletion characteristics. FIG. 3A shows graphs illustrating the length distribution of small deletion characteristics in the GLASS cohort. The upper panel graphs compare mean deletion lengths in primary vs. recurrent IDH mutant glioma (n=81), separated by RT-treatment (RT−, n=49; RT+, n=32). Statistical testing, paired Wilcoxon signed-rank test. A significant increase in mean deletion lengths was only observed for ionizing radiation treated samples. The lower panel graph shows deletion proportions calculated for each patient, comparing Primary and Recurrence in non-hypermutant glioma treated with RT (n=44). Y-Axis, proportion of deletions; X-Axis, deletion length>1 bp; mean (point) and 95% CI (line-range). Statistical testing, paired Wilcoxon signed-rank test (*=p<0.05, **=p<0.01). FIG. 3B shows graphs illustrating the deletion length distribution in the metastatic cohort. The boxplots of the upper panel graph compare mean deletion lengths in RT-naïve (RT−), RT+ pal, and RT+ cur samples. Kruskal-Wallis test was applied for statistical testing. The lower panel graph shows deletion proportions calculated for each patient, comparing RT-naïve (RT−), RT+ pal, and RT+ cur samples. Y-Axis, proportion of deletions; X-Axis, deletion length>1 bp; mean (point) and 95%-CI (line-range). Statistical testing, Kruskal-Wallis test (*=p<0.05, **=p<0.01, ***=p<0.001, ****=p<0.0001). In lower panel: RT−, diamond; RT+ pal, dot with black border and light gray fill; RT+ cur, dark dot. FIG. 3C shows graphs depicting relations to genomic features in the GLASS cohort. The upper panel forest plot shows distributions of deletions in relation to genomic features. Y-Axis, non-B-DNA genomic feature; X-Axis, log 10 ratio of mean distance of non-radiation-associated and radiation-associated post-treatment deletions to genomic feature over background distribution in non-hypermutated glioma samples (n=69). Distribution of radiation-associated deletions showed little variability (narrow 95% CI) and resembled background distribution more closely (closer to 0). Significant differences between radiation-associated deletions and non-radiation-associated deletions were seen in relation to repeats (Mann-Whitney Utest). In upper panel: RT−, light gray dot with asterisk; RT+, dark dot. The lower panel line graph shows the empirical cumulative distribution function (ECDF, Y-Axis) of distance to non-B-DNA features in kb (X-Axis), revealing a right-shift towards larger distances in post-radiated non-hypermutated recurrent samples (n=44). Longitudinal differences were not observed in either hypermutated or RT-naïve non-hypermutated glioma samples (additional data shown in FIG. 4C). In lower panel: A-phased repeat, lines with filled circles; direct repeat, lines with open squares; G-quadruplex motif, lines with filled squares; inverted repeat mirror, lines with open circles; repeat, lines with X's; short tandem repeat, lines with open triangles; and Z-DNA motif, lines with asterisks. Solid lines (all instances), primary; dashed lines (all instances), recurrence. FIG. 3D shows a schematic and graph illustrating categorization of small deletions in the GLASS cohort. The schematic (upper panel) depicts separating small deletions in the GLASS cohort into three major categories: 1 bp, >1 bp without microhomology, and >1 bp with microhomology in IDH mutant gliomas (n=81). The microhomology category was further classified based on the occurrence of microhomology repeat sequences and length of repeats. The graph (lower panel) compares the proportion of deletions for each non-hypermutated glioma sample treated with RT (n=44, further comparisons shown in Supp. FIG. 2E) using the paired Wilcoxon signed-rank test. Deletions of 1 bp length significantly decreased, whereas deletions>1 bp without microhomology significantly increased in response to RT. In FIG. 2D: >1 bp no microhomology (MH), n=3,325; 1 bp, n=11, 303; and >1 bp with MH, n=4,325.

FIG. 4A-F provides graphs illustrating comparisons of deletion data in various samples. FIG. 4A compares mean deletion lengths of newly acquired deletions (post-treatment fraction) in RT− vs RT+ IDHmut glioma samples. Statistical testing, Mann-Whitney U test. FIG. 4B shows mean deletion lengths in RT-naïve (RT−), palliative RT-treated (RT+ pal), and curative RT-treated (RT+ cur) tumor samples separated by primary tumor location in the metastatic cohort. Statistical testing, Kruskal-Wallis test. FIG. 4C shows longitudinal comparisons of mean distances of deletions of non-B DNA features in kb (X-Axis) in IDHmut glioma cases (Y-Axis). Cases were separated by radiation treatment status and hypermutation status. Note that neither in hypermutated, nor in RT-naïve non-hypermutated glioma samples significant longitudinal differences were observed. Statistical testing, paired Wilcoxon signed-rank test. FIG. 4D shows gene-wise dN/dS estimates by radiation treatment (rows) and fraction (columns) in the GLASS cohort. Genes are sorted by Q value (Bonferroni adjusted P value) and P value; Q values are indicated with bars. A vertical line indicates the Q value threshold of 0.05. No genes showed significant selection in the post-radiation fraction. FIG. 4E compares the proportion of deletions for IDHmut glioma samples separated by radiation treatment and hypermutation using the paired Wilcoxon signed-rank test. For each sample, the proportion of deletions with 1 bp length, >1 bp length with microhomology, and >1 bp length without microhomology add up to 1. [The lower right three panels (RT+ non-hypermutators) are reproduced from FIG. 2D for comparison with other groups.] 1 bp deletions were significantly increased in hypermutated radiation-naïve cases. No significant differences were observed for radiation-naïve non-hypermutated cases. FIG. 4F shows comparisons of deletion proportions in the metastatic cohort between RT-treated (RT+ pal and RT+ cur) and RT-naïve (RT−) cases using the Kruskal-Wallis test. In bone/soft tissue, breast, head and neck, and nervous system cancers, significantly lower proportions of deletions>1 bp with microhomology were observed in RT-treated samples compared to RT-naïve samples. In contrast, RT-treated breast, colon/rectum, esophagus, nervous system, and prostate tumor samples showed significantly higher proportions in deletions>1 bp without microhomology.

FIG. 5A-B provides graphs illustrating ID8 and APOBEC-SBS signatures associated with radiotherapy. FIG. 5A shows indel (ID) and single base substitution (SBS) mutational signatures in the GLASS cohort associated with RT (radiotherapy), hypermutation (HM), microsatellite instability (MSI), and homologous recombination deficiency (HRD). FIG. 5B shows indel (ID) and single base substitution (SBS) mutational signatures in the HMF cohort associated with RT (radiotherapy), hypermutation (HM), microsatellite instability (MSI) and homologous recombination deficiency (HRD). Statistical testing for both FIG. 5A and FIG. 5B applied the Mann-Whitney U test and false discovery rate (FDR) correction was used to adjust for multiple testing. Lightest-colored bars in petal plots did not reach statistical significance (defined as FDR<0.01).

FIG. 6A-F shows graphs illustrating aspects of indel burden following RT treatment and comparing indel signatures. FIG. 6A-D shows graphs depicting distributions of indel types for post-treatment mutations in the GLASS cohort, separated by RT status [FIG. 6A and FIG. 6C, RT-negative (RT−); FIG. 6B and FIG. 6D, RT-treated (RT+) and hypermutator (HM) status (FIG. 6A and FIG. 6B, HM; FIG. 6C and FIG. 6D, Non-HM)]. Patterns of indels in hypermutated samples resembled the previously identified MSI signature ID2, whereas RT-treated Non-Hypermutant gliomas harbored large similarities with ID8. Sample sizes for each subgroup are annotated. FIG. 6E shows graphs depicting a comprehensive comparison of all 17 COSMIC indel (ID) signatures in IDHmut gliomas, including absolute and relative signature contributions. The first set of graphs displays longitudinal comparisons of absolute signature contributions separated by radiation treatment status (RT− and RT+), and primary (Prim) vs. recurrence (Rec.) is evaluated for each. The second set of graphs displays longitudinal comparisons of relative signature contributions separated by radiation treatment radiation treatment status (RT− and RT+), and primary (Prim) vs. recurrence (Rec.) is evaluated for each. The paired Wilcoxon signed-rank test was applied for statistical testing for the first and second sets of graphs. The third set of graphs shows boxplots comparing absolute (upper row of panels) and relative (lower row of panels) signatures of post-treatment indels between RT-naïve (RT−) and RT-treated (RT+) samples. The Mann-Whitney U test was applied for statistical testing for the third set of graphs (ns=not significant, *=p<0.05, **=p<0.01, ***=p<0.001, ****=p<0.0001). ID8 was the only signature consistently associated with radiation therapy across different comparisons, nominating it as a robust signature of radiotherapy. FIG. 6F shows boxplots depicting the absolute and relative contributions of ID8 signature in metastatic cohort compared between cases with prior radiation treatment (RT+ pal, palliative; RT+ cur, curative) and cases without prior radiation treatment (RT−) separated by tumor types. Most tumor types showed significantly higher values of the signature in curative RT+ cases. Kruskal-Wallis test was applied for statistical testing.

FIG. 7A-C presents graphs illustrating RT-associated structural variations. FIG. 7A shows association of RT with increases in large deletions and inversions in an analysis of structural variants (SVs) after RT in IDHmut glioma samples with sufficient quality for calling (n=70). For each patient, the number of SVs were calculated pre-and post-treatment. The proportion of samples with or without increase of given SVs between RT-treated (RT+) vs RT-naïve (RT−) were compared. Based on the distribution of percent increase from primary to recurrence, the cutoff was set for a >50% increase (as shown in FIG. 8A). Dark shaded bar, increase >50%; light shaded bar, increase not >50%. Statistical testing, Fisher's exact test. FIG. 7B shows proportions of IDHmut glioma samples (n=81) harboring a homozygous deletion in CDKN2A, illustrating association of RT with CDKN2A homozygous deletions. Using Fisher's exact test, proportions were compared between RT-received recurrence (RT+) vs. RT-naïve recurrence (RT−) and RT-received recurrence (RT+) vs. samples prior to treatment (Primary). (Detailed distributions of whole chromosome deletion scores are shown in FIG. 8F). FIG. 7C shows violin plots illustrating RT-associated whole chromosome aneuploidy. The upper panels show longitudinal comparisons of whole chromosome aneuploidy scores separated by RT-treatment for IDHmut glioma samples with sufficient quality for calling and complete treatment annotation (total n=69, RT-treated n=42, RT-naïve n=27). The lower panels show separation of whole chromosome aneuploidy into whole chromosome gain (left two panels) and whole chromosome loss (right two panels) scores, respectively. The increase of whole chromosome aneuploidy in RT-treated samples was associated with whole chromosome losses. Dots are proportional to the frequency of whole chromosome loss integer for each subgroup. Paired Wilcoxon rank-signed test was applied for statistical testing. FIG. 7D shows graphs illustrating validation of SV and aneuploidy results in the metastatic cohort. The upper panels show violin plots comparing whole chromosome deletion scores between RT-naïve (RT−) vs RT+ pal vs RT+ cur and/or CDKN2A homdel vs. WT samples. CDKN2A homdel was associated with higher whole chromosome deletion scores, independent of RT. Within samples with CDKN2A homdel, samples that were RT-treated with curative intent showed the highest deletion scores. Dots are proportional to the frequency of whole chromosome loss integer for each subgroup. Statistical testing, Kruskal-Wallis test; detailed distributions of whole chromosome deletion scores are shown in Supp. FIG. 8G. The lower panel shows a multivariable Poisson regression model for whole chromosome deletion scores integrating RT, CDKN2A, and tumor types as variables. Curative radiotherapy and CDKN2A homozygous deletion were independently associated with higher levels of whole chromosome deletions.

FIG. 8A-G presents graphs illustrating analyses of associations of structural variants (SV) with RT and a schematic diagram. FIG. 8A shows an analysis of structural variants (SVs) in glioma samples (Translocations, Duplications, Deletions, and Inversions). For each patient, the number of SVs were calculated pre-and post-treatment and the proportional increase after therapy for each SV type was plotted separately for RT-naïve (RT−) and RT-treated (RT+) samples. Based on the distribution of proportional increase from primary to recurrence, a cutoff was defined for >50% increase that was further used for analyses (FIG. 7A). Supporting the analyses shown in FIG. 7A, FIG. 8B shows a multivariable logistic regression model fitted for the >50% increase values of the structural variant types, including radiation therapy, TMZ therapy, molecular subtype, and surgical interval as variables. Radiation therapy was independently associated with an increase in large deletions and inversions, but not duplications and translocations. FIG. 8C shows a schematic overview of separation of aneuploidy events into whole chromosome aneuploidy as a result of simple segregation errors and partial aneuploidy as a result of complex segregation errors. FIG. 8D shows violin plots of longitudinal analysis of partial aneuploidy in IDHmut glioma samples. Neither the general partial aneuploidy values (upper panels), nor the detailed separation of partial aneuploidy values into gain of chromosome arms (chromosome arm gain/neutral, lower left panels), loss of chromosome arms (chromosome arm loss/neutral, lower middle panels), and complex chromosome arm alterations (chromosome arm gain/loss, lower right panels) showed significant differences for any radiation treatment group. Dots are proportional to the frequency of whole chromosome loss integer for each subgroup. Statistical testing, paired Wilcoxon rank-signed test. FIG. 8E shows a multivariable Poisson regression model for whole chromosome losses in IDHmut glioma including molecular subtype, RT, TMZ, surgical interval, and CDKN2A status at recurrence as variables. A CDKN2A homdel, but not RT, was independently associated with higher whole chromosome losses. FIG. 8F shows density plots over integers of whole chromosome deletion scores for comparison between primary vs. recurrent glioma samples, separated by radiotherapy. In plots: primary, line with S's; recurrence, solid line. FIG. 8G shows density plots over integers of whole chromosome deletion scores for comparison between RT-naïve (RT−) vs RT+ pal vs RT+ cur and/or CDKN2A homdel vs. wild-type (WT) samples from the HMF dataset. A CDKN2A homdel was associated with higher whole chromosome deletion scores, independent of RT. Within samples with a CDKN2A homdel, samples that were RT-treated with curative intent (RT+ cur) showed the highest deletion scores. In upper graphs: CDKN2A homdel, line with X's; CDKN2A wild-type (WT), solid line. In lower graphs: lower graphs: RT−, line with asterisks; RT+ pal, solid line; RT+ cur, line with open diamonds.

FIG. 9A-B presents graphs illustrating survival probabilities for small deletion burdens. FIG. 9A shows associations of RT-related deletions with survival in the GLASS cohort. Samples were separated into three tertiles based on deletion burden at recurrence: high [(n=16) medium dark line, top tertile], intermediate [(n=16) lightest line, middle tertile], and low [(n=17, darkest line, bottom tertile]. Dotted lines indicate median overall survival times. Tertiles showed a stepwise association with survival. The left panel shows Kaplan-Meier survival plots comparing overall survival dependent on deletion burden at recurrence in RT-treated IDH mutant glioma samples (n=49 with available survival information), using the log-rank test. The middle graph shows Kaplan-Meier survival plots comparing surgical interval/time to second surgery dependent on deletion burden at recurrence using the log-rank test. The graph on the right shows Kaplan-Meier survival plots comparing post-recurrence survival dependent on deletion burden at recurrence using the log-rank test. FIG. 9B shows associations of RT-related deletions with survival in the metastatic cohort, using Kaplan-Meier survival plots comparing survival time dependent on deletion burden at metastasis in RT-treated metastases (n=958 with available survival information), using the log-rank test. Samples were separated into three tertiles based on deletion burden: high [(n=16), dark grey line with open triangles, top tertile], intermediate [(n=16) light grey line, middle tertile] and low [(n=17) dark grey line, bottom tertile). Dotted lines indicate median survival times. Tertiles showed a stepwise association with survival.

FIG. 10A-C presents graphs illustrating associations of CDKN2A status, aneuploidy burden, and ID8 burden with reduced survival. FIG. 10A shows a Kaplan-Meier survival plot (upper panel) comparing overall survival time dependent on CDKN2A status at recurrence in IDH mutant glioma samples, using the log-rank test. The lower panel shows a multivariable Cox regression model including the following variables: CDKN2A status at recurrence, TMZ treatment status, molecular subtype, and age. In plot upper panel: CDKN2A WT, dark gray line; CDKN2A homdel, dark gray line with open triangles; P<1.0e-04. FIG. 10B shows graphs comparing survival time dependent on various burdens at metastasis. The first graph shows a Kaplan-Meier survival plot comparing survival time dependent on CDKN2A status at metastasis in RT-treated metastases (n=958 with available survival information), using the log-rank test. The second graph shows a Kaplan-Meier survival plot comparing survival time dependent on aneuploidy burden at metastasis in RT-treated metastases (n=958 with available survival information), using the log-rank test. Samples were separated into three tertiles based on whole chromosome loss aneuploidy scores: high [(n=319), dark gray line with open triangles; top tertile]; intermediate [(n=319), light gray line, middle tertile], and low [(n=320), dark gray line, bottom tertile]. P=1.9e-04. The third graph shows a Kaplan-Meier survival plot comparing survival time dependent on RT signature ID8 burden at metastasis in RT-treated metastases (n=958 with available survival information), using the log-rank test. Samples were separated into three tertiles based on ID8 burden: high [(n=319), dark gray line with open triangles; top tertile], intermediate [(n=319), light gray line, middle tertile], and low [(n=320), dark gray line, bottom tertile]. P=4.0e-03. A low ID8 burden was associated with better survival, indicating a better response to RT. FIG. 10C shows a multivariable Cox regression model in RT-treated IDH mutant samples including deletion burden at recurrence as a continuous variable, and variables for CDKN2A homozygous deletion, TMZ treatment, molecular subtype, and age.

### DETAILED DESCRIPTION

The invention provides predictive and prognostic parameters to assist in tailoring the treatment of cancer in individual patients, also referred to herein as “subjects'” with this disease. The invention, in part, provides methods that can be used to assist in selecting treatments for, and for treating individual subjects who have been administered a radiotherapy. Methods of the invention can be used to determine effects of the radiotherapy on somatic DNA in the subject, which can be used to assist in selecting suitable therapies with which to treat a subsequent cancer in the subject. In addition, certain embodiments of methods can be used to determine effects of radiotherapy administered to a subject on somatic DNA in cells of the subject. Effects of administered radiotherapy in a subject can be used to assess the subject's risk if diagnosed with a subsequent cancer and/or to assess the subject's risk of mortality from a cancer.

It has now been identified that a significant increase of small DNA deletions may occur in cells in a subject following administration of ionizing radiation (also referred to herein as radiotherapy or radiation therapy) to the subject. Certain embodiments of methods of the invention include analyzing one or more characteristics of small deletions resulting from the radiotherapy administered to a subject and comparing the subject's results to control results can assist in determining a treatment for a subject following the radiotherapy. As described herein, it has now also been identified that the small deletion signatures can be associated with genomic alterations, which permits characteristics of the small deletions to determine a prognosis for a subject who has had cancer, and to help predict an outcome of a cancer in the subject. Changes in characteristics of small deletions, plurality of small deletions, and small deletion signatures may be referred to here as “worsening” or “worse.” A worsening of a small deletion signature may be said to be worse, or to have worsened, if one or more characteristics of the small deletions have changed in a manner that indicates a radiotherapy administered to a subject resulted in one or more of: an increase in a number of small deletions, an increase in sizes (on average) of small deletions in a plurality of small deletions, an increase in the small deletion burden in a subject, and the presence and/or size of small deletions in a following radiotherapy as compared to a pre-radiotherapy sample or other control sample. A subject whose post-radiotherapy small deletion signature is determined to be worse than one or more of the subject's pre-radiotherapy small deletion signature and a “healthy” control signature. The term, “small deletion signature” may be used interchangeably with the term “small deletion burden,” when used in reference to assessment of small deletions in a subject's DNA.

Some embodiments of methods of the invention include determining one or more characteristics of a plurality of small deletions DNA sequence in a biological sample obtained from a subject. As used herein the term “plurality” means, two or more and in some embodiments a plurality is 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more small deletions per megabase of DNA sequence. Characteristics of small deletions resulting from radiotherapy that may be determined using method of the invention, include but are not limited to: small deletion length, small deletion distribution, genomic signatures of the small deletions, presence or absence of a plurality of small deletions, quantity of the small deletions in a plurality of small deletions, and genetic identity of one or more of a plurality of the small deletions.

### Small Deletion Signatures and Burden

Embodiments of methods of the invention can be used to identify and use a small deletion signature for a subject. A small deletion signature may be based on a small deletion burden identified for the subject. Identification of the subject's small deletion burden may be done by assessing one or more characteristics of somatic DNA in a biological sample obtained from the subject. As detailed elsewhere herein, characteristics assessed may include one of more of: small deletion sizes, relative amount of small deletions, absolute amount of small deletions, locations of small deletions, etc. Methods of the invention can be used to determine at least one characteristic of a plurality of small deletions in somatic DNA in a biological sample obtained from the subject. In some embodiments, methods of the invention are used to determine one or more characteristics, such as but not limited to: number of small deletions and sizes of small deletions in the sample obtained from the subject following administration of a radiotherapy to the subject to treat a cancer in the subject. Results of this post-radiotherapy determination can be used to select a treatment regimen for the subject and the selected treatment regimen may be administered to the subject following administration of a radiotherapy to the subject to treat a cancer in the subject, and the results used to identify the subject's prognosis with respect to recurrence and/or progression of a cancer in the subject, and the results can also be used to identify a likelihood of the subject's mortality from a cancer. Thus, a post radiotherapy assessment of small deletions in a biological sample obtained from a subject following radiotherapy treatment for a cancer can be used to select later treatment regimens, identify a prognosis for a later cancer in the subject, and to help determine a risk of the subject's mortality from a later cancer.

A subject diagnosed with a cancer may be administered therapies such as, but not limited to: surgery, radiotherapy, chemotherapy, etc. Following conclusion of a cancer therapeutic regimen, many subjects are at a later time again diagnosed with cancer. A later-diagnosed cancer in a subject, which is also referred to herein as a “subsequent” cancer, may be one or more of: a recurrence of the subject's previous cancer, a metastatic cancer arising from the subject's previous cancer, and a new cancer, whose origin appears distinct from the previous cancer. It has now been determined that a radiotherapy regimen administered to a subject can result in DNA changes in somatic cells in the subject, and identifying certain such changes can assist in selecting a therapeutic regimen for the subject in the event of a later-diagnosed cancer in the subject. For example, following radiotherapy administration to a subject, changes in somatic DNA of the subject may occur. Such changes may include, but are not limited to one or more of an increase in the number of small deletions in somatic DNA of the subject, an increase in the presence of small deletions of greater size present in somatic DNA of the subject, an increase in the small deletion burden in the DNA of the subject, and use of methods of the invention to assess one or more of these types of changes can assist in selecting a therapy for a subsequent cancer in the subject, and/or to determine a prognosis for a subsequent cancer in the subject, and/or to predict mortality of the subject from a subsequent cancer.

Characteristics of small deletions in DNA of a subject are referred to as the small deletion signature for the subject. A subject's small deletion signature can differ at different times. For example after being administered radiotherapy a subject may have a small deletion signature that is different from the same subject's small deletion signature prior to receiving the radiotherapy. The term “signature” as used herein in reference to small deletions resulting from radiotherapy administered to a subject, means characteristics of the small deletions present in DNA of somatic cells of the subject. A biological sample obtained from a subject prior to radiotherapy treatment may be assessed to determine a baseline or control small deletion signature for that subject and the control signature may be compared to a post-radiotherapy signature to assess changes in the subject's signature. Change or lack of change in a subject's small deletion signature following radiotherapy can be used in methods of the invention to help select a treatment for a subsequent cancer in the subject and to obtain prognostic and survival information in relation to a subsequent cancer in the subject.

Differences and/or changes between a subject's post-radiotherapy small deletion characteristics and a control, which can, but need not be, a control small deletion signature obtained from the subject prior to the radiotherapy, may include a change in the determined number of small deletions in the subject's sample. In some embodiments of the invention, the determined number of small deletions in the subject's post-radiotherapy sample is at least 10%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500% higher than a control number of small deletions. In such circumstances abstention from radiotherapy may be selected as a treatment for the subject in the event of a subsequent cancer. In certain embodiments of the invention, a determined number of small deletions in a subject's post-radiotherapy sample is at least 0.25, 0.5, 1, 2, 3, 4, 5, 6, 7, 8, 9, 10-fold higher than a control number of small deletions. In such circumstances abstention from radiotherapy may be selected as a treatment for the subject in the event of a subsequent cancer.

With respect to prognosis and mortality assessment, an embodiment of a method of the invention can be used to determine a small deletion signature or small deletion burden and if the results indicate a worsening of the small deletion burden a prognosis for the subject in the event of a subsequent cancer may be determined to be worse versus the prognosis in the absence of a worse post-radiotherapy small deletion signature/small deletion burden. The term prognosis as used herein to indicate the physiological state of a subject with respect to the cancer. A subject with a poor prognosis may be a subject whose cancer progresses and worsens. A poor prognosis may also correlate to an increased likelihood of risk of death of the subject from the subsequent cancer. As a non-limiting example, a biological sample is obtained from a subject who has received a radiotherapy for a cancer and the small deletion burden determined for the subject. The small deletion results from the post-radiotherapy biological sample are compared to small deletion burden results from a biological sample obtained from the subject prior to receiving the radiotherapy. A statistically significant increase in small deletion burden in the post-radiotherapy biological sample compared to the pre-radiotherapy biological sample identifies a poorer prognosis for the subject compared to the subject's prognosis if the result of the post-radiotherapy small deletion burden indicates no increase or a decrease in the subject's small deletion burden compared to the pre-radiotherapy small deletion burden results. It will be understood that a pre-radiotherapy small deletion burden of a subject may be used as a control small deletion burden in a method of the invention to assess prognosis of a subject, but a control small deletion burden used may also be a control result based on small deletion burden results obtained from one or a plurality of other subjects. Use of controls and control results is routinely practiced in the art and additional details of certain embodiments of controls are provided elsewhere herein.

Similarly, an embodiment of a method of the invention can be used to determine a small deletion burden and if the results indicate a worsening of the small deletion burden a risk of mortality for the subject may be identified as having a higher risk of mortality from a cancer than would be the case in the absence of the worse post-radiotherapy small deletion burden. As a non-limiting example, a biological sample is obtained from a subject who has received a radiotherapy for a cancer and the small deletion burden determined for the subject. The small deletion results from the post-radiotherapy biological sample are compared to small deletion burden results from a biological sample obtained from the subject prior to receiving the radiotherapy. A statistically significant increase in small deletion burden in the post-radiotherapy biological sample compared to the pre-radiotherapy biological sample identifies a higher risk of mortality for the subject compared to the subject's mortality risk if the result of the post-radiotherapy small deletion burden indicates no increase or a decrease in the subject's small deletion burden compared to the pre-radiotherapy small deletion burden results. It will be understood that a pre-radiotherapy small deletion burden of a subject may be used as a control small deletion burden in a method of the invention to assess risk of mortality, but a control small deletion burden used may be one determined based on one or a plurality of other subjects. Use of controls and control results is routinely practiced in the art and additional details of certain embodiments of controls are provided elsewhere herein.

In some embodiments of the invention, a characteristic of the plurality of small deletions is small deletion burden in a subject. The term “small deletion burden” refers to the quantity and/or type of small deletions in a subject's small deletion signature and thus includes small deletion characteristics such as, but not limited to: a number of small deletions, location of small deletions, and size of small deletions. In some embodiments of the invention, the determined small deletion burden in a subject's post-radiotherapy sample is at least 10%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500% higher than a control small deletion burden, and may include any percentage within the stated range. In some embodiments, a determined small deletion burden in a subject's post-radiotherapy sample is between 10% and 50%, 10% and 100%, 10% and 200%, 10% and 500%, 20% and 50%, 20% and 100%, 25% and 55%, 25% and 100%, 25%, and 500%, 30% and 60%, 30% and 100%, 30% and 500%, 40% and 50%, 40% and 100%, 50% and 100%, 60% and 200%, 60% and 500%, 80% and 200%, 80%, and 500%, 100% and 200%, 100%, and 500%, 200%, and 500% higher than a control small deletion burden. In such circumstances abstention from radiotherapy may be selected as a treatment for the subject in the event of a subsequent cancer.

In non-limiting examples, a determined small deletion burden in a subject's post-radiotherapy sample that is greater than 25%, 30%, 40%, 50%, 60%, 70%, 80%, 90%, 100%, 150%, 200%, or higher compared to a control small deletion burden, such as but not limited to the subject's pre-radiotherapy small deletion burden, results in selection of abstention of radiotherapy as a treatment for the subject in the event of a subsequent cancer. In certain embodiments of the invention, a determined small deletion burden in a subject's post-radiotherapy sample is at least 0.25, 0.5, 1, 2, 3, 4, 5, 6, 7, 8, 9, 10-fold higher than a control small deletion burden. In such circumstances abstention from radiotherapy may be selected as a treatment for the subject in the event of a subsequent cancer. In such circumstances a prognosis for the subject in the event of a subsequent cancer may be worse and the risk of mortality from a subsequent cancer may be determined to be higher in the subject versus the prognosis and risk of mortality in the absence of a worse post-radiotherapy small deletion signature.

In some embodiments of the invention, a characteristic of the plurality of small deletions is the length of small deletions in a plurality of small deletions in a subject. Lengths of small deletions may be between 5 and 100 base pairs (bp). In certain embodiments, lengths of the small deletions in a plurality of small deletions are between 5 and 50 bp. In some embodiments, lengths of the small deletions in a plurality of small deletions are between 5 and 15 bp. In some embodiments, lengths of small deletions in a plurality of small deletions are less than 100 bp in length. It will be understood that a plurality of small deletions may include small deletions of two or more different lengths. In some embodiments of the invention, determined sizes of small deletions in a subject's post-radiotherapy sample are on average, at least 10%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500% higher than sizes in a control. In such circumstances abstention from radiotherapy may be selected as a treatment for the subject in the event of a subsequent cancer. In some embodiments, a determined sizes of small deletions in a subject's post-radiotherapy sample is between 10% and 50%, 10% and 100%, 10% and 200%, 10% and 500%, 20% and 50%, 20% and 100%, 25% and 55%, 25% and 100%, 25%, and 500%, 30% and 60%, 30% and 100%, 30% and 500%, 40% and 50%, 40% and 100%, 50% and 100%, 60% and 200%, 60% and 500%, 80% and 200%, 80%, and 500%, 100% and 200%, 100%, and 500%, 200%, and 500% larger than a control sizes of small deletions. In such circumstances abstention from radiotherapy may be selected as a treatment for the subject in the event of a subsequent cancer, and the subject may receive a treatment that includes abstention from radiotherapy.

In certain embodiments of the invention, determined sizes of small deletions in a subject's post-radiotherapy sample are at least 0.25, 0.5, 1, 2, 3, 4, 5, 6, 7, 8, 9, 10-fold higher than a control small deletion sizes. In such circumstances abstention from radiotherapy may be selected as a treatment for the subject in the event of a subsequent cancer. In such circumstances a prognosis for the subject in the event of a subsequent cancer may be worse and the risk of mortality from a subsequent cancer may be determined to be higher in the subject versus the prognosis and risk of mortality in the absence of a worse post-radiotherapy small deletion signature.

In some embodiments of methods of the invention, a biological sample obtained from a subject is assessed for small deletion burden and the results compared to a certain threshold, which in some embodiments is an absolute change (a non-limiting example of which is +0.5 small deletions/Mb) and in certain embodiments is a relative change (a non-limiting examples of which are 50% additional new small deletions and/or a size change of 5-fold), compared to a control. In some embodiments, a control value is a small deletion burden and/or sizes of small deletions determined by a method of the invention to assess a biological sample obtained from the subject in advance of receiving a radiotherapy. In some embodiments, a control is a result obtained from a plurality of other biological samples/subjects. Use of controls is well-known in the art and further information about controls is provide elsewhere herein.

In some embodiments of methods of the invention, gene locations of small deletions are determined as a characteristic of a plurality of small deletions in somatic DNA in a biological sample obtained from a subject. Some embodiments of methods of the invention include assessing whether or not the small deletion burden of a subject is associated with mutations in selected genes. Non-limiting examples of genes that can be assessed are: ATM, ATR, CHEK1, CHEK2, PARP1, PRKDC, TP53 and WEE1. These genes are involved in the DNA damage response (DDR). It has been identified that mutations in the DDR are associated with a significantly higher small deletion burden. In some embodiments of methods of the invention, one or more of the genes: ATM, ATR, CHEK1, CHEK2, PARP1, PRKDC, TP53 and WEE1 are assessed using a method of the invention and the number and/or length of deletions in the gene(s) is determined. In some embodiments of methods of the invention deletions and chromosomal loss are assessed in CDKN2A. In some embodiments of methods of the invention, CDKN2A status and aneuploidy burden are assessed with respect to small deletion status.

Additional details regarding assessing small deletion signatures, characteristics of small deletions, and their use in selecting a treatment for a cancer, determining a cancer prognosis, and/or determining a mortality risk for a subject are provided in the drawings, drawing descriptions, and Examples herein.

### Radiotherapy

A subject diagnosed with a cancer may receive one or more radiation treatments as at least a part of their cancer therapy. In some instances, a subject diagnosed with a cancer is treated with a radiation regimen that includes one or a plurality of radiation administrations to the subject as a treatment for the cancer. It will be understood that an initial radiation regimen may be followed by one or more subsequent radiation regimens if it is determined necessary for the subject. Non-limiting examples of radiation regimens for cancer in a subject are: five radiation administrations to the subject per week for three, four, five, six, seven, eight, or nine weeks; two radiation administrations per week for five weeks; and one radiation administration. It will be understood that a cancer treatment regimen may include different parameters of such as: the amount of radiation administered, the frequency of radiation administration, and the number of administrations of radiation to the subject in the treatment regimen. An initial radiation regimen may be administered to a subject following an initial identification of a cancer in the subject and a subsequent radiation regimen may be administered to the same subject following a subsequent identification in the subject.

The term “radiotherapy” as used herein to refer to a single administration of radiation to a subject or to refer to a regimen of two or more radiation treatments administered to a subject to treat the subject's cancer. It will be understood that the term “regimen” as used means one or a plurality of radiation treatments prescribed to a subject to treat a cancer in the subject. For example, upon a diagnosis of a cancer in a subject, a healthcare professional may prescribe a regimen of one or more radiation administrations to treat the cancer in the subject. In some instances a pre-radiotherapy small deletion signature may be determined in a sample obtained from a subject prior to a single radiation treatment and/or regimen of radiation treatments. In some embodiments of the invention a pre-radiotherapy small deletion signature of a subject is used as a control small deletion signature for that subject. In some embodiments of the invention, a post-radiotherapy small deletion signature may be determined for a subject following a single radiation treatment or a regimen of radiation treatments and one or more characteristics of small deletions in the post-radiotherapy biological sample can be compared to the one or more characteristics of small deletions in the pre-radiotherapy biological sample obtained from the subject (e.g., the subjects own “control” sample), or can be compared to a control small deletion signature's one or more characteristics of small deletions based on one more biological samples not specific to the subject.

### Treatment Methods and Compounds

Certain aspects of the invention include selecting methods and compounds to treat a subsequent cancer in a subject. Non-limiting examples of cancer treatments are: surgery, radiotherapy, chemotherapy, administration of a pharmaceutical agent, immunotherapy, modified T cell therapy, virus-based therapy, abstention from surgery, abstention from chemotherapy, and abstention from administration of a pharmaceutical agent. Treatments for a cancer in a subject that may be selected based at least in part on results obtained using methods of the invention, include, but are not limited to abstention from radiotherapy. In a non-limiting example, a small deletion signature is determined for a subject diagnosed with a recurrent cancer, a subsequent cancer, a metastasis of a previous cancer, and compared to a control small deletion signature. If the comparison indicates one or more characteristics of the subject's small deletion signature has statistically significantly worsened compared to the control signature, treatment selected for the recurrent, subsequent, metastatic cancer in the subject comprises abstention from radiotherapy. It will be understood that the treatment selected may include one or more of: surgery, chemotherapy, administration of a pharmaceutical agent, an immunotherapy, a modified T cell therapy, a virus-based therapy, abstention from surgery, abstention from chemotherapy, and abstention from administration of a pharmaceutical agent. It will be understood that radiotherapy may be delivered using various art-known means or forms, non-limiting examples of which are: external beam radiation and brachytherapy. The term radiotherapy may be used herein in reference to palliative radiotherapy and in reference to a curative radiotherapy. In some embodiments, a radiotherapy administered to a subject is a palliative radiotherapy. It will be understood that the term “palliative radiotherapy” means a radiation therapy administered to a subject to do one or more of: shrink a cancer; shrink a tumor; slow a cancer's growth; slow a tumor's growth; stop or slow progression of a cancer or tumor; and stop, slow, or reduce symptoms caused by the cancer or tumor. Palliative radiotherapy may be administered to a subject to reduce focal symptoms of advanced cancer, either symptoms arising from a primary tumor or one or more metastatic growths in the subject. In some embodiments, palliative radiotherapy comprises administration of high energy X-rays to a focused region in the subject, for example a tumor site in the subject. In certain embodiments, a radiotherapy administered to a subject is a curative radiotherapy. A curative radiotherapy may include radiation administered to a subject that is one or more of: more broadly administered to the subject, more frequently administered to the subject, and at a higher dose level of radiation delivered to a subject, compared to a palliative radiotherapy. A subject may be administered one or more different forms and may be administered 1, 2, 3, 4, 5, 6, 7, different administrations of a radiotherapy.

In another non-limiting example, a small deletion signature is determined for a subject diagnosed with a recurrent cancer, a subsequent cancer, a metastasis of a previous cancer, and compared to a control small deletion signature. If the comparison indicates one or more characteristics of the subject's small deletion signature has not statistically significantly worsened compared to the control signature, treatment selected for the recurrent, subsequent, metastatic cancer in the subject may comprise radiotherapy. It will be understood that a treatment selected in this situation may include one or more of: radiotherapy, surgery, chemotherapy, administration of a pharmaceutical agent, an immunotherapy, a modified T cell therapy, a virus-based therapy, abstention from surgery, abstention from chemotherapy, and abstention from administration of a pharmaceutical agent.

### Treatment Selection

Using certain embodiments of methods of the invention, one or more small deletion characteristics determined for a subject can be used to select a treatment regimen for the subject. Characteristics of small deletions and small deletion signatures can be compared to control characteristics and signatures to determine one or more differences between the compared characteristics. For example, a subject's post-radiotherapy small deletion signature shows that the subject has one or more of: a higher number of small deletions than the subject's pre-radiotherapy small deletion signature; larger sizes of small deletions than the subject's pre-radiotherapy small deletion signature, etc., it supports a therapeutic decision to abstain from further a radiotherapy in that subject. Similarly, for example, a higher number of small deletions and/or larger sizes of small deletions determined to be present in a subject's post-radiotherapy small deletion signature compared to the subject's pre-radiotherapy small deletion signature, indicates a worse prognosis and/or greater mortality risk from cancer for the subject, than is indicated in the absence of these small deletion signature differences. In some instances, a control number of small deletions is zero and if the determination of a number of small deletions in a subject's post-radiotherapy sample is statistically significantly greater than zero, the selected treatment comprises abstaining from administering a radiotherapy to the subject.

Following administration of a radiotherapy to that subject, a small deletion signature is again determined for the subject but this time using a biological sample obtained from the subject after the radiotherapy. In some embodiments, a post-radiotherapy biological sample is obtained at least 1, 2, 3, 4, 5, 6, 7, 8, 9, 10, 15, 20, 25, 30, 35, 40, 45, 50, 55, 60, 65, 70, 75, 80, 85, 90, 95, or 100 days after administration of a radiotherapy to the subject. In some embodiments, a post-radiotherapy biological sample is obtained at least 1, 2, 3, 4, 5, 6, 7, 8, 9, 10, 15, 20, 25, 30, 35, 40, 45, 50, 55, 60, 65, 70, 75, 80, 85, 90, 95, or 100 weeks after administration of a radiotherapy to the subject. In some embodiments, a post-radiotherapy biological sample is obtained from a subject at least 1, 2, 3, 4, 5, 6, 7, 8, 9, 10, 20, 30, 40, 50, 60, 70, 80, 90, or 100 months after administration of a radiotherapy to the subject. In some embodiments, a post-radiotherapy biological sample is obtained at least 1, 2, 3, 4, 5, 6, 7, 8, 9, 10, 20, 30, 40, 50, 60, 70, 80, or 90 years after administration of a radiotherapy to the subject.

### Assays and Assessments

Methods of the invention, in some aspects, are used to assess and compare small deletion characteristics in a sample comprising a plurality of cells. In some embodiments, a sample is a biological sample obtained from a subject. DNA sequencing methods can be used in certain embodiments of the invention to determine at least one characteristic of a plurality of small deletions in a biological sample. Non-limiting examples of DNA sequencing methods that can be used in embodiments of the invention include, but are not limited to: exome sequencing and whole genome sequencing. These and other sequencing methods suitable for use in methods of the invention are known and routinely practiced in the art.

Some embodiments of the invention include use of a sequencing means to determine one or more characteristics of small deletions in a biological sample. In some instances sequencing results indicate “presence” of small deletions in a sample. In certain instances sequencing results indicate “absence” of small deletions in a sample. In certain embodiments, methods of the invention include determining a size of one or more small deletions in a plurality of small deletions. In addition, in certain embodiments of the invention, a nucleic acid sequence identity of one or more small deletions in a plurality of small deletions is determined.

Methods set forth in certain embodiments of the invention can be used in conjunction with art-known sequencing and sequence-identification methods to identify the status of one or more characteristics of a plurality of small deletions in a biological sample, and to evaluate and compare one or more characteristics of a small deletion signature of a subject versus a control small deletion signature. Methods of the invention, in some embodiments, include determining one or more characteristics of a plurality of small deletions in somatic DNA in a sample and comparing the determined characteristics of a control. In some embodiments of the invention methods such as one or more of: microarray analysis, deep sequencing, polymerase chain reaction (PCR), real time PCR, northern blotting, in situ hybridization RNA-seq, and qPCR, may be used to determine characteristics of small deletion signatures.

### Controls

In some embodiments of the invention, methods may include comparing a characteristic of small deletions in somatic DNA in a sample to a control value of the characteristic of the small deletions in somatic DNA. As used herein a “control” may be a normal control or a control known to have a certain small deletion characteristics. In some embodiments, a normal control is a small deletion signature that does not indicate that radiotherapy resulted in sufficient small deletion characteristics to select abstention of radiotherapy treatments in a subsequent cancer. A normal control may be obtained from historical databases of characteristics of small deletions and small deletion signatures in biological samples obtained from one or a plurality of subjects. In some embodiments of the invention, a control small deletion characteristic is the determined characteristic in a biological sample, cell(s), and/or one or a plurality of subjects. Means of selecting and using appropriate controls in comparative, diagnostic, treatment, and assay methods are well known in the art. In some embodiments of the invention, a normal control is prepared from a biological sample obtained from a subject prior to a radiotherapy administration to the subject. Thus, in some embodiments of the invention, a control for a subject reflects a baseline value for that subject because the sample used to determine the control values is obtained from the subject prior to the subject receiving radiotherapy for a cancer in the subject.

A control characteristic for small deletions can be readily be determined by measuring one or more characteristics in a sample using a method of the invention, as described herein or other art-known means. In some embodiments of the invention, a control level of one or more small deletion characteristics is based on small deletion characteristics determined in a plurality of subjects, or from a single subject.

### Subject, Diseases, Cells, and Samples

As used herein, a subject shall mean a vertebrate animal including but not limited to a human, mouse, rat, guinea pig, rabbit, cow, dog, cat, horse, goat, and primate, e.g., monkey. In certain aspects of the invention, a subject may be a domesticated animal, a wild animal, or an agricultural animal. Thus, the invention can be used to test for and treat diseases or conditions in human and non-human subjects. For instance, methods and compositions of the invention can be used in veterinary applications as well as in human treatment regimens. In some embodiments of the invention, the subject is a human. In some embodiments of the invention, a subject has a cancer.

A subject at risk of having or suspected of having a cancer is a subject who has been diagnosed with a cancer or is believed likely to have a cancer based on factors such as clinical examination, symptoms, and other art-known methods to assess cancers. For example, though not intended to be limiting, visual and/or physical examination of a subject may suggest the subject as likely to have a cancer. Art-known diagnostic procedures and assessments can be used to determine if a subject is a risk of having, is believed to have, is likely to have, or is diagnosed as having a cancer. Methods of the invention can be used to determine a treatment for, determine a prognosis for, and determine a risk of mortality from numerous types of cancers, non-limiting examples of which are: a brain cancer, a neuroblastoma, a glioma, a bone cancer, a soft-tissue cancer, a breast cancer, a colon cancer, a rectal cancer, an esophageal cancer, a head and neck cancer, a lung cancer, an ovarian cancer, a prostate cancer, a skin cancer, a urinary tract cancer, a central nervous system (CNS) cancer, and a uterine cancer. In some embodiments of the invention a brain cancer is a glioma.

Cells that may be assayed and/or treated using methods and compounds of the invention include but are not limited to mammalian cells, human cells, vertebrate cells, non-human mammalian cells, cultured cells, tumor cells, somatic cells, etc. A sample of the invention may be referred to as a biological sample, and may include, for example, a sample obtained directly from a subject, a sample of cells obtained from a subject and stored and/or cultured, or other suitable sample. A biological sample may include biological material, non-limiting examples of which are one or more of: tissue, cells, blood, serum, saliva, cerebrospinal fluid (CSF) fluid, and lymph fluid. Non-limiting examples of biological samples that can be obtained from a subject assessed using an embodiments of a method of the invention is a tissue sample, a tumor sample, a biopsy obtained from a subject, and a circulating blood sample obtained from a subject. In a non-limiting example, circulating tumor DNA (ctDNA) is isolated from a biological sample comprising blood or cerebrospinal fluid of a subject using commercially available methods such as from Qiagen (Qiagen, Germantown, MD). The ctDNA is them used to identify tumor-type-specific signatures. The burden of small deletions, a normalized approximation of the total number of small deletions across the genome, is compared to the burden of small deletions detected in the genome of a tumor specimen. When the positive difference between ctDNA-derived and tumor specimen-derived small deletion burden exceeds a certain threshold, which is either an absolute (+0.5 small deletions/Mb) or a relative increase (50% additional new small deletions), it may be decided that additional treatment regimens based on ionizing radiation will not be effective. Thus, information obtained from assessing the burden of small deletions in a subject is used to select a treatment regimen for the subject, and the subject is administered the selected treatment regimen.

In some embodiments of the invention, a biological sample comprises cancer cells obtained from the subject. Routine procedures can be used to obtain samples for use in methods of the invention and to carry out pre-testing steps such as separation, purification, or other routine preparation steps. As used herein the term “sample” and “biological sample” may be used interchangeably. In some embodiments of the invention a sample may comprise one or more of cells and tissues obtained from a subject. One or more samples may be obtained from a subject using art-known methods, non-limiting examples of which are: resection, biopsy, blood draw, fluid draw, scraping, punch biopsy, needle biopsy, fluid collection, and surgical removal. Obtaining samples from subjects is routinely practiced in the art and art-known methods can be used in conjunction with the information provided herein.

### Kits

The invention also encompasses kits for detecting one or more characteristics of small deletion signatures. For example, a kit can comprise one or more reagents and solutions that can be used for one or more of: full genome sequencing, exome sequencing, nucleic acid detection, sequence identification, etc. and that are capable of aiding in determining one or more characteristics of small deletions in a sample. Characteristics such as, but not limited to: presence or absence of small deletions, size of small deletions, small deletion burden, etc. can be determined using elements provided in an embodiment of a kit of the invention. Solutions and reagents can be packaged in suitable containers. The kit can also include a means for comparing one or more characteristics determined in a sample with the one or more characteristics in a standard or control and/or can also include instructions for using the kit to determine one or more characteristics of small deletion signatures and use of the determinations to assist in selecting a treatment. A kit of the invention may also include one or more of a: detectable label, enzyme, buffer, container, and other items for use in carrying out methods of the invention.

A kit of the invention may also include instructions. Instructions typically will be in written form and will provide guidance for carrying out the preparation and procedure for one or more methods of the invention.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1 Radiotherapy Treatment is Associated with an Increased Small Deletion Burden

### Materials and Methods

The methods described herein apply to Example 1 and subsequent Examples as appropriate.

### Patient Cohort

A cohort of 190 patients with high-quality longitudinal DNA sequencing data was curated, including treatment naïve primary and matched post-treatment first recurrence tumor samples from the GLASS dataset [Barthel, F. P. et al., Nature 576, 112-120 (2019)]. Paired samples were classified into three subtypes according to the 2016 World Health Organization (WHO) classification: IDH mutant with 1p/19q co-deletion (IDHmut-codel), IDH mutant without 1p/19q co-deletion (IDHmut-noncodel), and IDH wild type (IDHwt) [Louis, D. N. et al., Acta Neuropathol. 131, 803-20 (2016)]. The GLASS cohort used herein consisted of n=106 whole genome sequencing (WGS) samples (n=53 primary samples, n=53 matched first recurrence samples) and n=274 whole exome sequencing (WES) samples (n=106 primary samples, n=106 matched first recurrence samples). Detailed information on sequence platforms, capture kits, and read length information were as previously described [Barthel, F. P. et al., Nature 576, 112-120 (2019)].

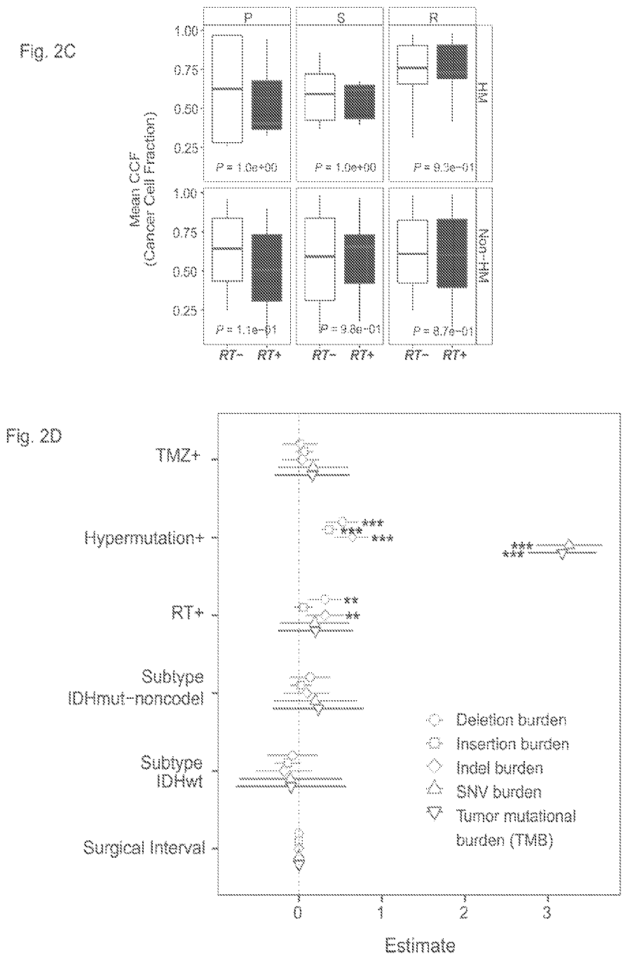

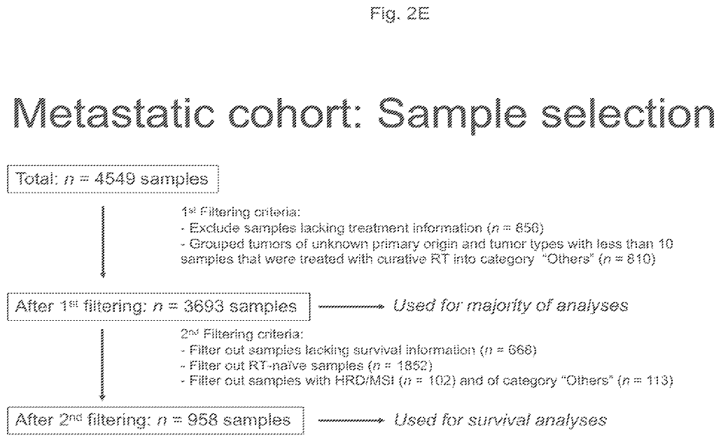

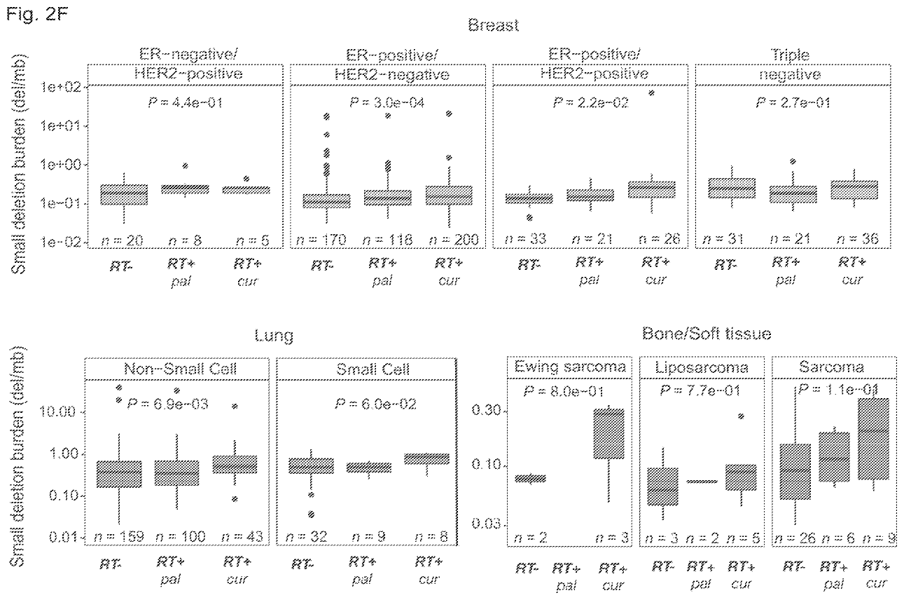

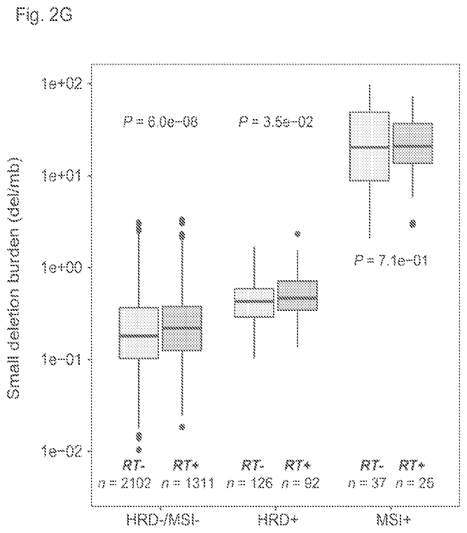

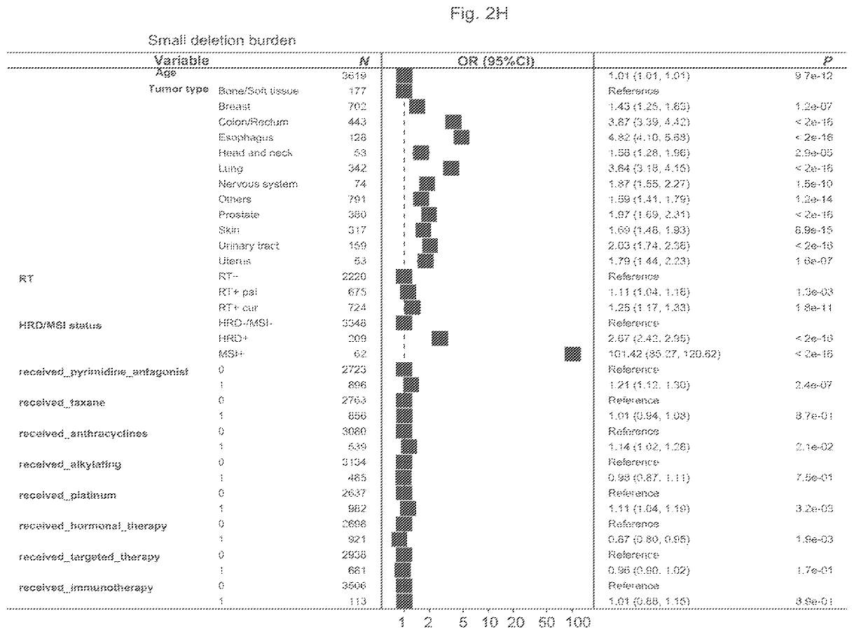

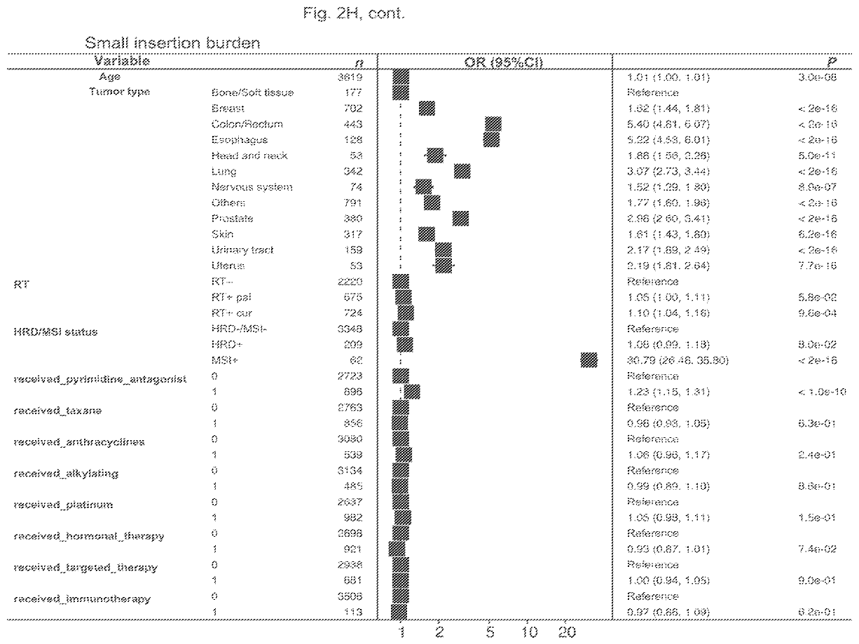

For validation analyses, a metastatic cohort from the Hartwig Medical Foundation (HMF; Amsterdam, NL) was curated, comprising a total of 4,549 samples [Priestley, P. et al., Nature 575, 210-216 (2019)]. The HMF cohort consisted of metastatic tumor samples collected following local or systemic treatment as part of the CPCT-02 (NCT01855477) and DRUP (NCT02925234) clinical trials. Biopsy samples from a wide range of tumor types collected at various hospitals across the Netherlands were sequenced at the core facilities of the Hartwig Medical Foundation. Whole genome sequencing (WGS) was performed for each sample according to standardized protocols [Bins, S. et al., Oncologist 22, 33-40 (2017)]. Detailed information on sequence platforms, capture kits, and read length information were as previously described [Priestley, P. et al., Nature 575, 210-216 (2019)]. VCF files with mutations and associated metadata were downloaded from The Hartwig Medical Database (database.hartwigmedicalfoundation.nl). After application of filtering criteria (as described in FIG. 2E) a set of n=3,693 samples were defined and used for the majority of analyses described below herein. For survival analyses, curative RT-treated samples with sufficient survival information (n=958) were selected. All prior radiotherapy data were extracted using clinical data as present in the CPCT-02 eCRF on Dec. 8, 2020. These data were not cleaned and represented the data entered by the clinical sites. The prior radiotherapy was categorized as curative intent, palliative intent, or other. All other instances were manually curated. All adjuvant/neo-adjuvant or post-operative radiotherapy was considered curative intent radiotherapy. All local radiotherapy for pain relief or other symptom-directed goals were considered as palliative. Some items were not specified, and those events were not included in the analysis. All radiotherapy for non-malignant disease states was excluded, specifically for gynecomastia treatment after castration. Over- or underrepresentation of the radiation signatures could not be excluded as it was not known whether the metastases that were biopsied were not already present at the time of radiotherapy.

### Variant Calling

Variant calling in the GLASS dataset was performed according to the GATK Best practices using GATK 4.1.0.0 as previously described [Barthel, F. P. et al., Nature 576, 112-120 (2019)]. Briefly, GATK 4.1.0.0 was used for variant calling in tumor samples against a matched normal control. Additionally, panels of normals were constructed across multiple control samples from the same tissue source and sequencing center. Variants were broadly filtered for germline variants, cross-sample contamination, read orientation, and sequence context. Variants were called across all samples for a given patient. Variants with a minimum coverage of 10 reads in both primary and recurrence and a minimum VAF of 10% for either the primary or the recurrence were included for further analysis. Variants were considered to be present if at least one mutant read was detected in a sample. Mutations directly overlapping with known repeat regions according to the repeatmasker database were removed. Specifically, all variants in known repeat regions were filtered out, including DNA satellites, microsatellites, long terminal repeats, transposable elements (LINE/SINE elements), and low complexity regions. Variant clonality was inferred for each patient individually using PyClone (v.0.13.1) and as previously described [Barthel, F. P. et al., Nature 576, 112-120 (2019)]. Pipeline scripts can be found at github.com/fpbarthel/GLASS.

### Mutation Burden Comparison

The mutation burden was calculated as the number of mutations per megabase (Mb) with at least 10× coverage and stratified by variant type. The overall tumor mutation burden (TMB) was calculated as the sum of the burden of small deletions, small insertions, and single nucleotide variants. Recurrent tumors with greater than 10 mutations per Mb were considered hypermutated as previously described [Barthel, F. P. et al., Nature 576, 112-120 (2019)]. For the comparison of mutation burden between RT-treatment groups in the GLASS dataset, the burden of mutations unique to the recurrent tumors and therefore acquired after treatment was calculated. To adjust for confounding covariables, a multivariable log-linear regression model was fitted using the glm function in R. In addition to RT-treatment, TMZ-treatment, hypermutation, surgical interval in months, and molecular subtype were included as variables. The small deletion burden in the GLASS dataset was not confounded by batch effects. Accordingly, the full therapy and tumor type information was included for mutation burden analyses in the Hartwig metastatic cohort. To adjust for negative infinite values resulting from the log-transformation in the GLASS cohort, a constant value of 1 was added to the log function. For the metastatic cohort, the log-transformation did not result in (negative) infinite values and therefore did not necessitate the addition of a constant value.

### Statistical Methods

All data analyses were conducted in R 3.6.1 (broadly using tidyverse 1.3.0), Python 3.7.3, and PostgreSQL 10.5. R was interfaced with the PostgreSQL database used for data storage using the unixODBC 2.3.6 driver plus DBI 1.0.0 and odbc 1.1.6 R packages. All survival analyses including Kaplan-Meier plots and Cox proportional hazards models were conducted using the R packages survival and survminer. For unpaired group comparisons the Mann-Whitney Utest and Kruskal-Wallis test were used, and for paired longitudinal comparisons the Wilcoxon signed-rank test was applied. Forest plots were generated using the R package forestmodel. Survival times for the GLASS dataset were calculated as described previously [Barthel, F. P. et al., Nature 576, 112-120 (2019)]. In the HMF metastatic cohort, survival was calculated starting from the date of biopsy to date of death. For patients that were alive, the last date of follow-up (date of treatment end) was used as censoring.

### Data and Code Availability

Processed sequencing data from the GLASS project used in this and subsequent Examples are available at synapse.org/glass. Processed sequencing data from the Hartwig Medical Foundation (HMF) dataset used in this and subsequent Examples are available at hartwigmedicalfoundation.nl. The repeatmasker database used in this and subsequent Examples is available at repeatmasker.org/. Pipeline scripts used in this and subsequent Examples are available at github.com/fpbarthel/GLASS. Custom scripts for analyses performed in this and subsequent Examples are available at github.com/TheJacksonLaboratory/Radiation_Scars.

### Results

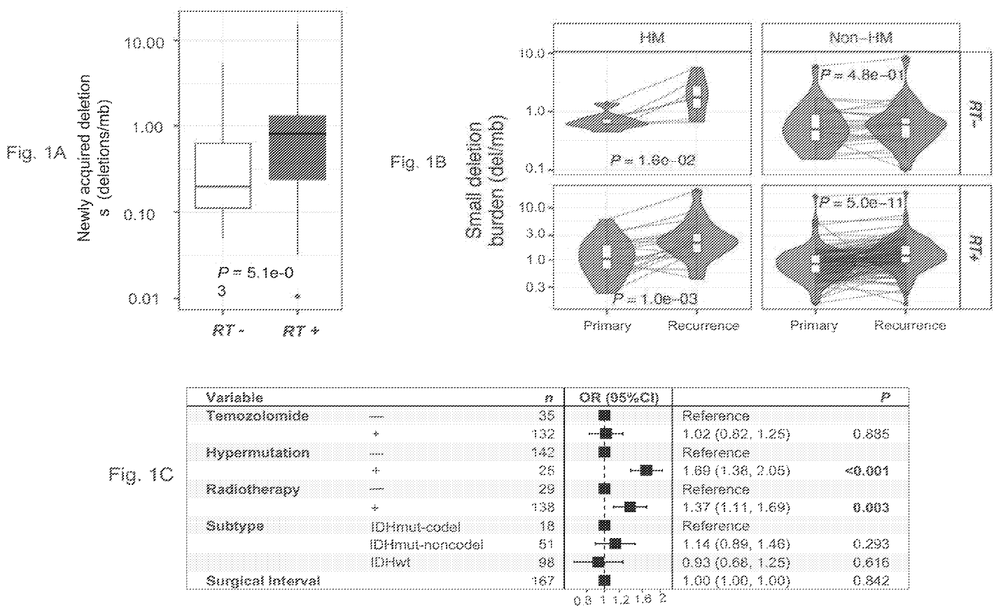

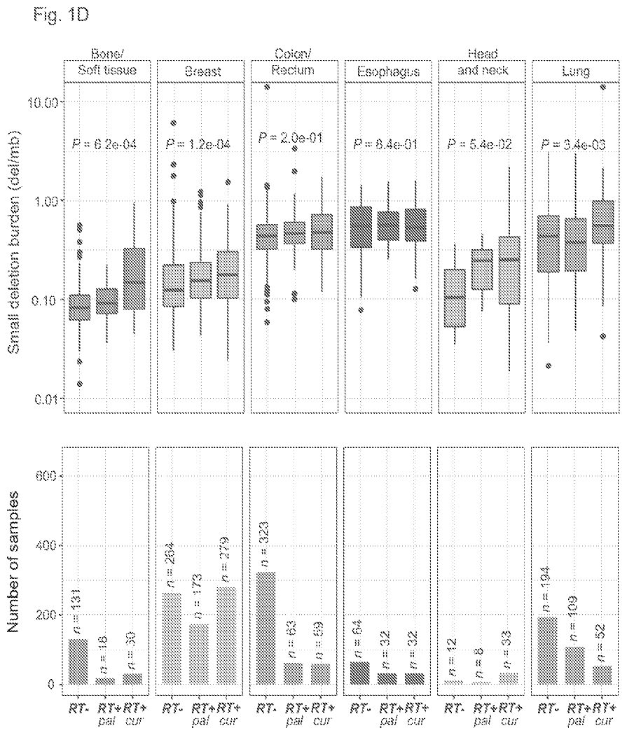

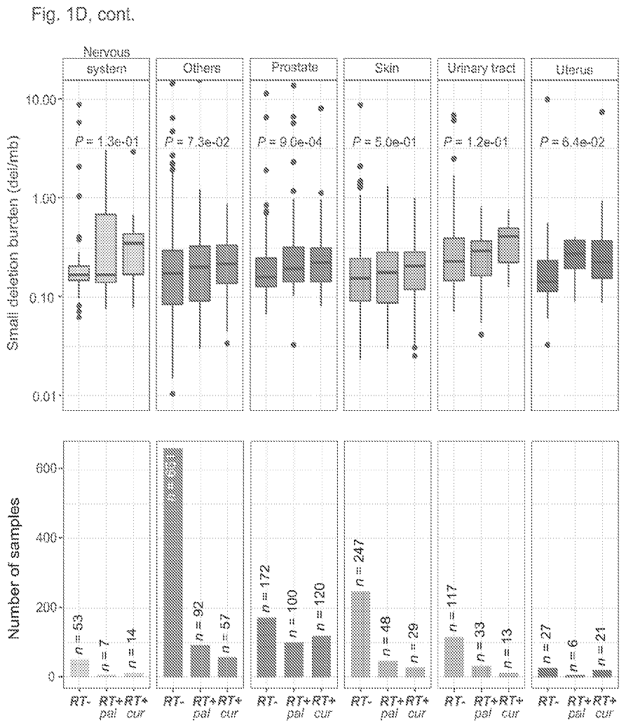

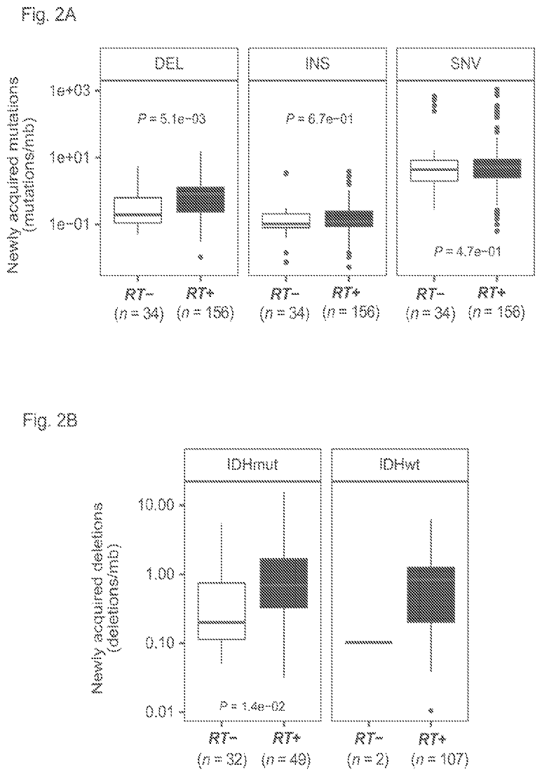

First, the contributions of radiotherapy (RT) and temozolomide (TMZ) on the burden of somatic mutations including small insertions/deletions (indels, length of 1 to 20 bp) and somatic single nucleotide variants (sSNVs) in the exomes of matched pre-and post-treatment glioma samples (n=190) were analyzed, of whom 119 (63%) cases had received the combination of RT and TMZ, 19 (10%) cases had received RT alone, 13 (7%) cases underwent TMZ treatment, and 16 (8%) cases had received no RT or TMZ treatment. For 23/190 (12%) cases, TMZ annotation was lacking, 18 of which had received RT. For each patient, mutations were separated into pre- (present in the initial tumor) and post-treatment (only present in the recurrent tumor). The mutation burden (average mutation frequency per megabase (Mb)) of post-treatment mutations was then calculated. A median of 0.68 new small deletions/Mb were acquired in recurrent RT-treated glioma which was significantly higher than the median of 0.19 new small deletions/Mb acquired in recurrent RT-naïve gliomas (RT−; FIG. 1A, P=5.1e-03, Mann-Whitney Utest), and significantly higher than the small deletion burden detected at diagnosis (FIG. 1). RT was not associated with a significant increase in the sSNV burden (FIG. 2A, P=4.7e-01, Mann-Whitney Utest) or small insertion burden (FIG. 2A, P=6.7e-01, Mann-Whitney U test). The increase in small deletions was particularly pronounced in the subset of gliomas marked by the presence of mutations in IDH1 or IDH2, a clinically relevant subtype [Louis, D. N. et al., Acta Neuropathol. 131, 803-20 (2016)] predominantly consisting of grade II and III gliomas (FIG. 2B, P=1.4e-02, Mann-Whitney Utest), while the number of RT-naive recurrent cases among IDH wild-type glioma was too small to test for differences (n=2, vs n=107 RT-treated cases). To ensure that these changes were not primarily due to TMZ-associated hypermutation (tumor mutation burden exceeding 10 mut/Mb at recurrence) [Barthel, F. P. et al., Nature 576, 112-120 (2019)], the cohort was stratified by hypermutation status. Hypermutation was associated with an increase in small deletions independent of RT-treatment, whereas amongst non-hypermutators only tumors from patients that received RT showed a significant increase in small deletions (FIG. 1B, P=5.0e-11, paired Wilcoxon signed-rank test), further implicating the observed increase in small deletions as a potential consequence of RT. To evaluate the independence of this finding from potential confounders, a log-linear regression model was fitted that included TMZ-treatment, glioma molecular subtype, time interval between surgeries, and hypermutation. RT was independently associated with an increase in small deletions (FIG. 1C, P=3e-03, t-test), directly attributing the observed increase in small deletions to RT-treatment. Cancer cell fractions were determined and it was found that post-treatment deletions in RT-treated patients did not show clonality differences compared to post-treatment deletions in RT-naïve patients, suggesting that these deletions were not more clonal/subclonal (FIG. 2C, hypermutant: P=9.3e-01, non-hypermutant: P=8.7e-01, Mann-Whitney Utest). Comparing the pre-treatment mutation burden and aneuploidy scores between glioma patients that acquired a high burden versus a low burden of post-treatment deletions revealed no significant differences, suggesting that these genomic characteristics of the pre-RT tumor are not predictive of the acquisition of small deletions in response to radiotherapy.

Importantly, 30% (41/136) of non-hypermutant samples gained more than 1 del/Mb following radiotherapy, whereas only 7% (2/27) of RT-naïve non-hypermutators acquired more than 1 del/Mb (P=1.6e-02, Fisher's exact test). Among the samples treated with ionizing radiation, 35% (55/156) showed a doubling of the small deletion burden when compared with the primary tumor. The effect of RT on mutational burden was significant for small deletions and not significant for other types of somatic mutations such as insertions and sSNVs (FIG. 2D). Conversely, TMZ-associated hypermutation was associated with significant increases in the burden of all types of mutations (FIG. 2D).

Following these observations, it was hypothesized that radiotherapy may similarly increase the number of small deletions in other tumor types. To test this hypothesis, whole-genome sequencing-derived mutational profiles were evaluated from 3693 metastatic tumors with complete treatment annotation (as described in FIG. 2E), available via the Hartwig Medical Foundation (hereafter “HMF” or “Hartwig”) dataset [Priestley, P. et al., Nature 575(7781), 210-216 (2019)]. Tumors were separated by site of origin and the small deletion burdens of RT-treated and naïve tumors were compared. RT-treated tumors were further stratified depending on whether the treatment intent was curative (RT+ cur, n=739) or palliative (RT+ pal, n=689), which generally differ in the applied cumulative dosage of ionizing radiation [Lutz, S. T. et al., J Clin. Oncol. 32, 2913-2919 (2014)]. While this analysis was restricted to single time-point mutational profiles, a significantly higher small deletion burden associated with curative RT was observed in multiple tumor types, including bone/soft tissue (RT+ cur: median 0.15 del/Mb, RT−: median 0.08 del/Mb, P=6.2e-04, Kruskal-Wallis test), lung (RT+ cur: median 0.56 del/Mb, RT−: median 0.43 del/Mb, P=3.4e-03, Kruskal-Wallis test), and breast (RT+ cur: median 0.18 del/Mb, RT−: median 0.12 del/Mb, P=1.2e-04, Kruskal-Wallis test) cancers (FIG. 1D). Further separation into tumor subtypes revealed that the observed patterns were present in both lung cancer types (FIG. 2F, Non-small cell lung cancer: P=6.9e-03 and small cell lung cancer: P=6.0e-0.2, Kruskal-Wallis test), but in breast cancer these were restricted to ER-positive subtypes (FIG. 2F, ER-positive/HER2-negative: P=3.0e-04 and ER-positive/HER2-positive: P=2.2e-02, Kruskal-Wallis test). Tumors treated palliatively with RT frequently presented an intermediate state in between the RT− and RT+ cur cohorts, suggesting an association between RT-treatment derived small deletion burden and RT dose exposure.

DNA repair deficient tumors had been shown to harbor an increased mutational load [Campbell, B. B. et al., Cell 171, 1042-1056.e10. (2017)]. To determine whether a DNA repair defective background had an impact on the small deletion burden in the HMF dataset, information on microsatellite instability (MSI) and homologous recombination deficiency (HRD) in the HMF dataset was derived from previous data [Nguyen, L. et al., Nat. Commun. 11, 5584 (2020)]. Notably, HRD+ and particularly MSI+ tumors harbored significantly more small deletions compared to samples that were HRD−/MSI− (FIG. 2G, P<2.2e-16, Kruskal-Wallis test). RT-treatment was associated with an increase in small deletion burden in HRD−/MSI− (FIG. 2G, P=6.0e-08, Mann-Whitney Utest) and HRD+ tumors (P=3.5e-02), but not in MSI+ tumors (P=7.1e-01). These results raised the possibility that DNA repair deficiencies like HRD and MSI confounded the association between RT-treatment and the small deletion burden. To address this, HRD and MSI status were included in the multivariable log-linear regression analysis, which showed that RT-treatment was associated with an increase in the small deletion burden independent of a number of potential confounders, including a DNA repair defective background (FIG. 2H).

The next assessment was whether the small deletion burden was associated with mutations in selected genes (ATM, ATR, CHEK1, CHEK2, PARP1, PRKDC, TP53 and WEE1) involved in the DNA damage response (DDR). This analysis indicated that DDR mutations were universally associated with a significantly higher small deletion burden. Log-linear regression was used to correct for potential confounding variables, including age, tumor type, DNA damage repair background, DRR gene mutations, and various cytotoxic treatment regimens (e.g. taxane, platinum, anthracyclines, alkylating agents) that have been previously associated with increased mutation burdens [Pich, O. et al., Nat. Genet. 51, 1732-1740 (2019)]. Results from this analysis revealed a robust association between both palliative and curative radiotherapy treatment but not any other therapy and small deletions (FIG. 2H, RT+ cur vs. RT-naïve: odds ratio=1.25, P<1e-03, t-test), confirming that the increased small deletion burden associated with radiotherapy was independent of tumor type, HRD, MSI, DDR gene mutations, or additional cytotoxic therapy.

To verify the causal association between RT and acquired small deletions, a previously published dataset [Kucab et al., Cell 177, 821-836.e16. (2019)] consisting of whole-genome sequencing data from 324 human-induced pluripotent stem cells (iPSCs) exposed to known or suspected environmental carcinogens, including two iPSCs treated with ionizing radiation, was re-analyzed. The small deletion burden was found to be significantly higher in the RT-treated iPSCs compared to controls (FIG. 2I, P=2.0e-02, Mann-Whitney U test). In contrast, no significant difference in small insertion burden was observed (P=1.8e-01). Strikingly, the ionizing radiation group showed the highest median burden of small deletions across all treatment modalities, further substantiating our human tissue analysis (FIG. 2J).

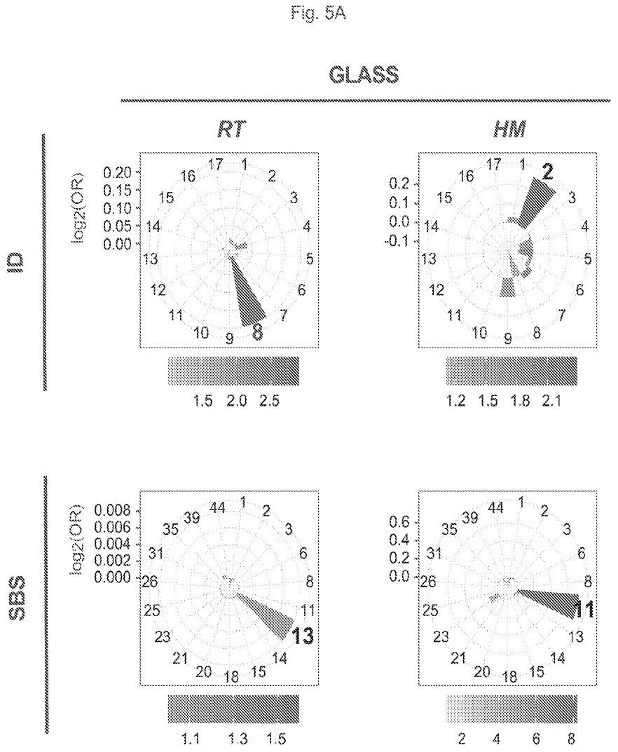

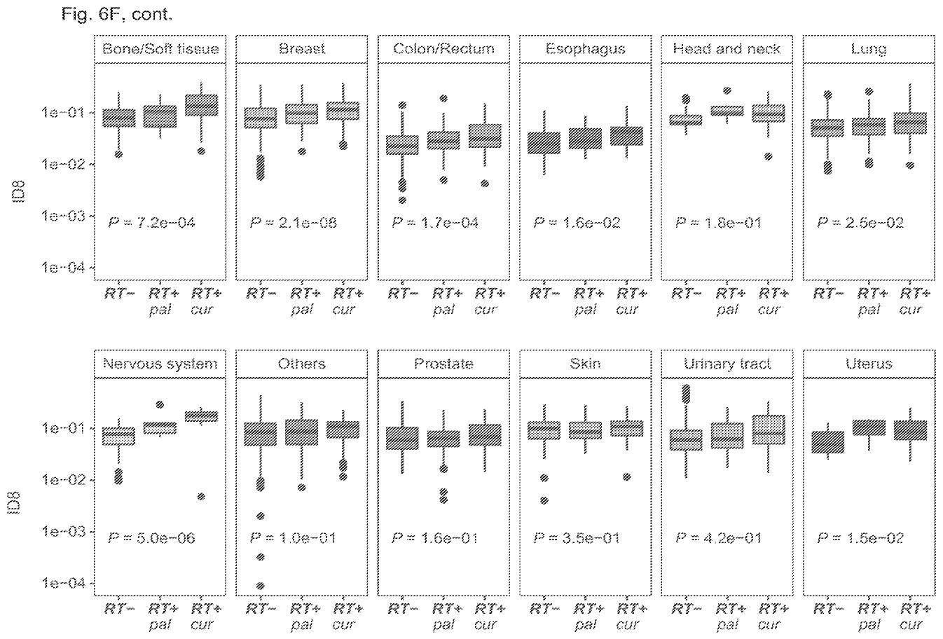

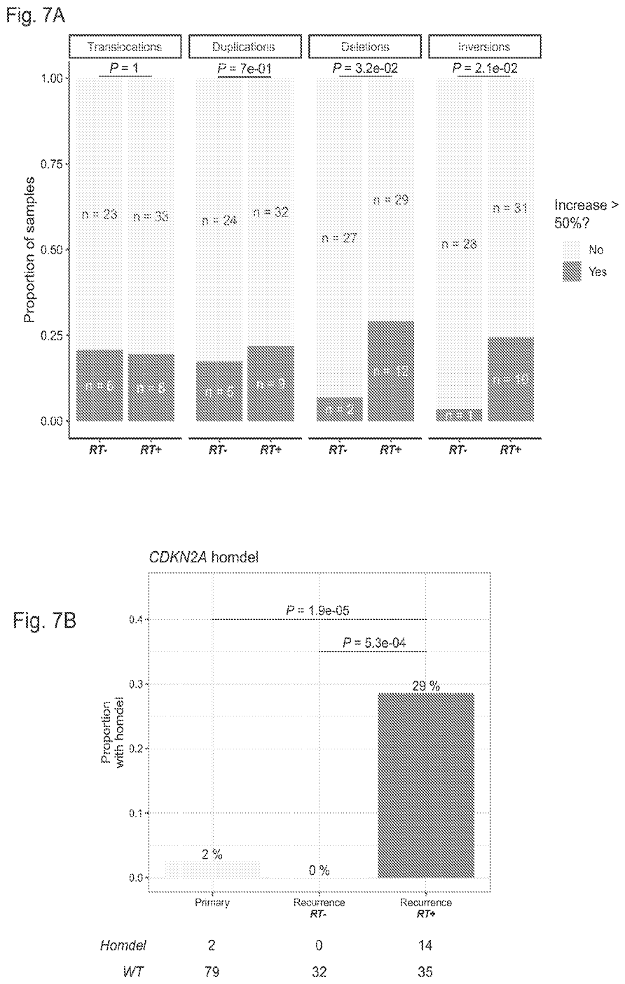

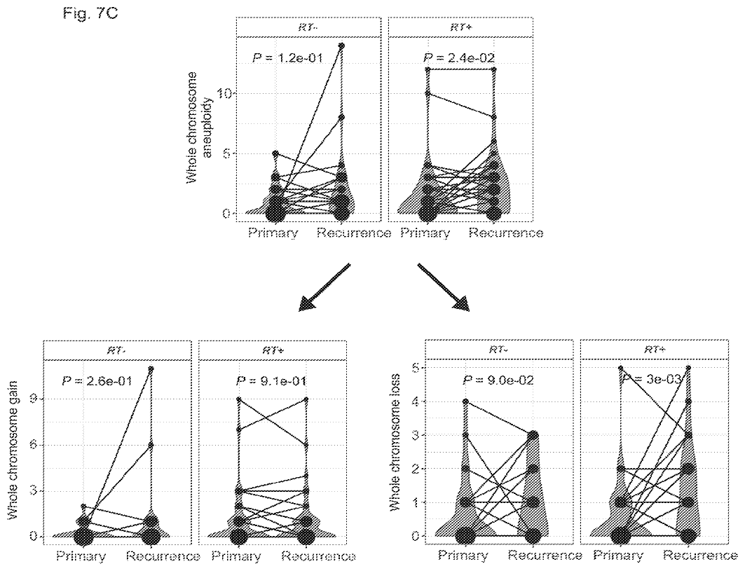

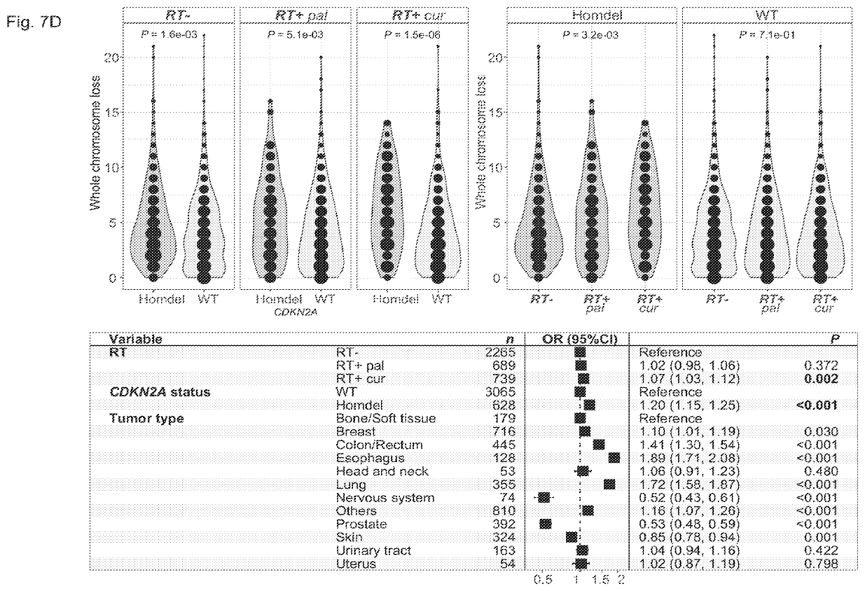

Pre- and post-treatment small deletions were compared to previously defined mutational indel signatures [Alexandrov, L. B., et al., Nature 578, 94-101 (2020)]. Indels from the GLASS cohort were separated into three fractions: private to primary (P, pre-treatment), shared between primary and recurrence (S, pre-treatment) and private to recurrence (R, post-treatment). For each fraction, the contribution of indel signatures was calculated and mean contributions between RT-treated and RT-naïve samples were compared. Indel signature 8 (ID8) had the highest contribution in the recurrence-only fraction (mean contribution=0.22) and was significantly higher in tumors from patients that received RT (P=1.68e-4, Q=8.56e-3, Mann-Whitney Utest and false discovery rate, respectively). Furthermore, in RT-treated patients but not RT-naïve patients (FIG. 7E) comparing ID8 values before and after treatment revealed significant increases in absolute (P=4.5e-07, Paired Wilcoxon rank-signed test) and relative (P=0.0023) ID8 contributions, respectively. Specifically, in the post-treatment fraction RT was significantly associated with ID8 (absolute ID8: P=2.3e-05, relative ID8: P=7.4e-05). Signature ID8 is composed of ≥5 bp deletions without microhomology and has previously been linked to DSB repair by c-NHEJ, providing further evidence that radiation-induced DSBs are primarily repaired via c-NHEJ. A previous analysis had indicated an increase in the proportion and frequency of ID12 signature mutations in the Hartwig dataset [Pich, O., et al., Nat. Genet. 51, 1732-1740 (2019)]. Directed by the instant findings in the GLASS cohort, an increase in ID8 mutations, not ID12, was the strongest and most significant association with radiotherapy treatment among metastatic tumors (FIG. 5A-B). Both absolute and relative ID8 values are significantly higher in RT-treated samples when compared to RT-naïve samples, and this association was independent of tumor type (FIG. 7F, FIG. 7G).

### Example 2 Radiotherapy-Associated Small Deletions Harbor a Characteristic Genomic

### Signature

### Materials and Methods

Materials and methods used were as described in Example 1 and below herein, as applicable.

Association of Deletions with Non-B DNA Structures

The genomic locations of non-canonical DNA structures were derived from the Non-B DNA database [Cer, R. Z. et al., Nucleic Acids Res. 41, D94-D100 (2013)]. For every variant position and, for comparison, for 250,000 randomly sampled positions from the reference genome, the distance to non-B features was calculated as a continuous (absolute distance to genomic feature in bp) or categorical (position in or up to 100 bp to genomic feature—yes/no) value. A Mann-Whitney Utest was used for differences in the genomic properties of variants in radiation-induced and non-radiation-induced tumors after adjusting for random background distribution.

dNdScv

For quantification of selection processes at the level of individual genes dependent on radiation therapy, dN/dS ratios were calculated as previously described [Barthel, F. P. et al., Nature 576, 112-120 (2019)]. Briefly, the R package dNdScv [Martincorena, I. et al., Cell 171, 1029-1041.e21 (2017)] was run using the default and recommended parameters for each mutational fraction (private to primary, shared between primary and recurrence, and private to recurrence). All analyses were conducted separately within radiotherapy-naïve and radiotherapy-treated groups.

### Sequence Microhomology

Sequence microhomology was determined by iteratively comparing the 3′ end of the deleted sequence to the 5′ flanking sequence. Any deletion demonstrating at least 2 nt of homology was considered microhomology-mediated. The homologous sequence was characterized and further analyzed for the presence of 1 nt, 2 nt, and 3 nt repeats. The repeat unit and number of repeats were quantified.

### Results

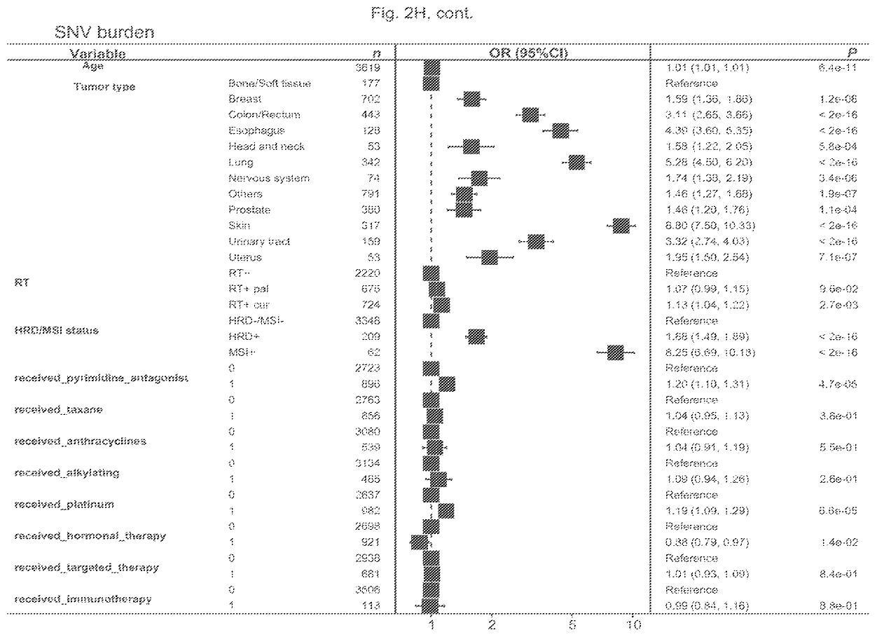

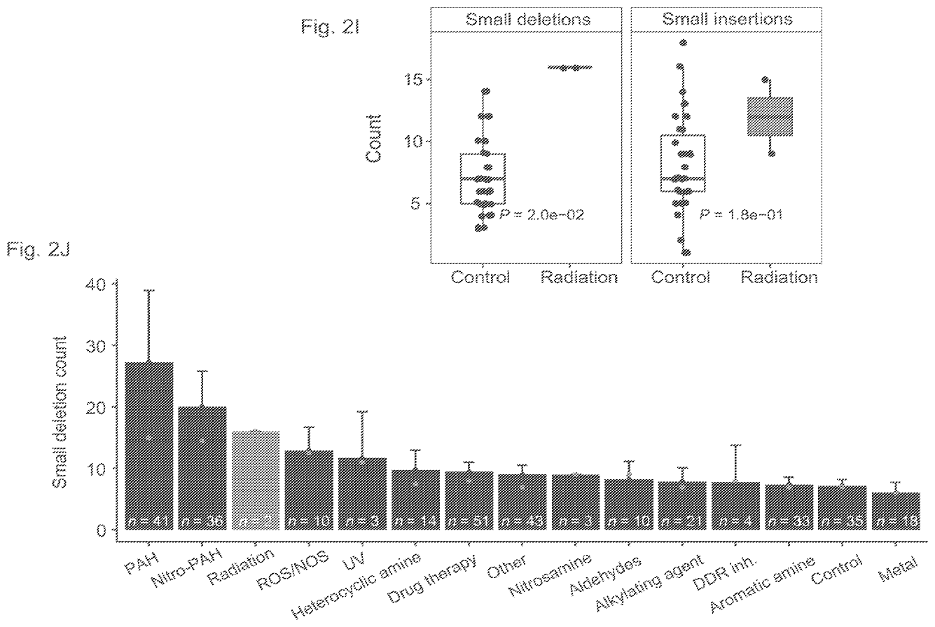

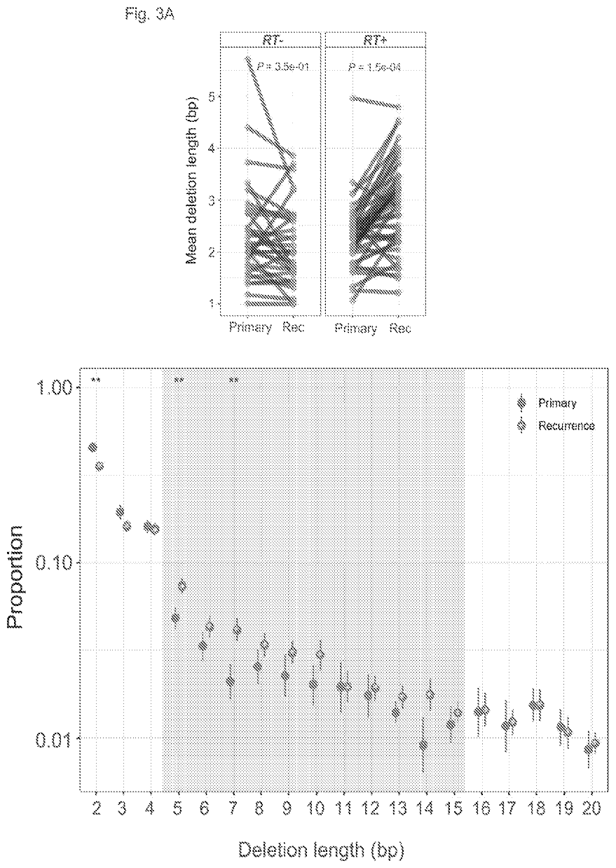

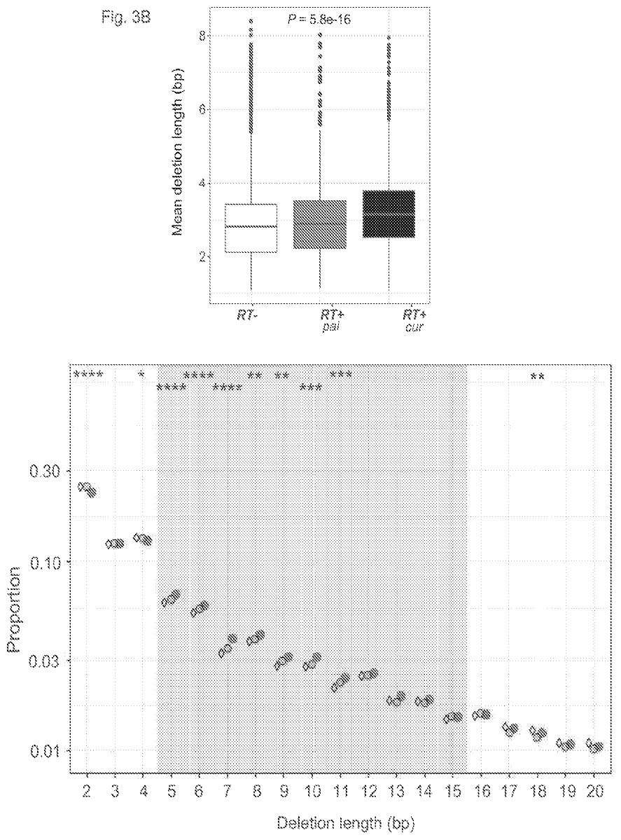

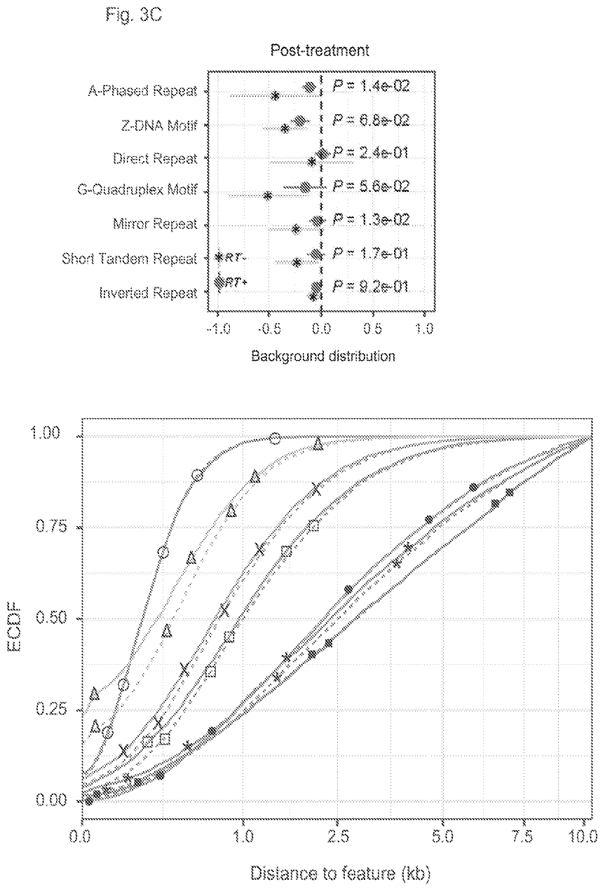

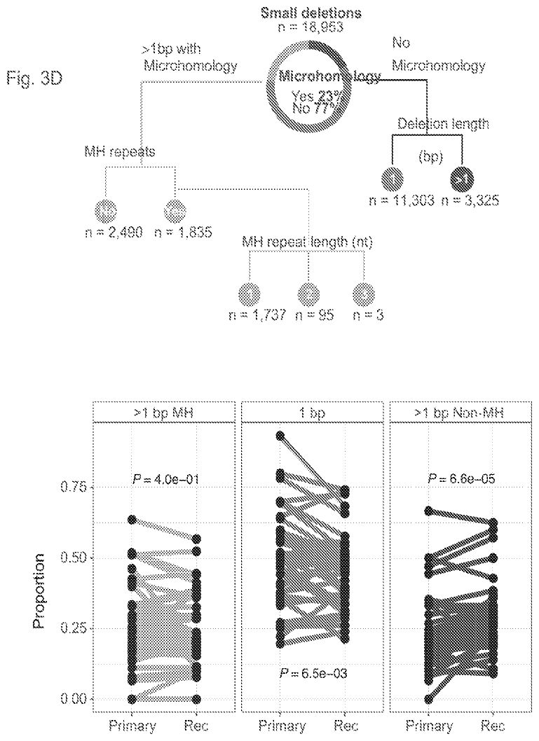

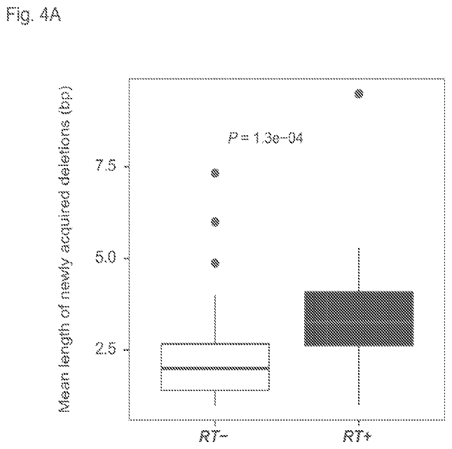

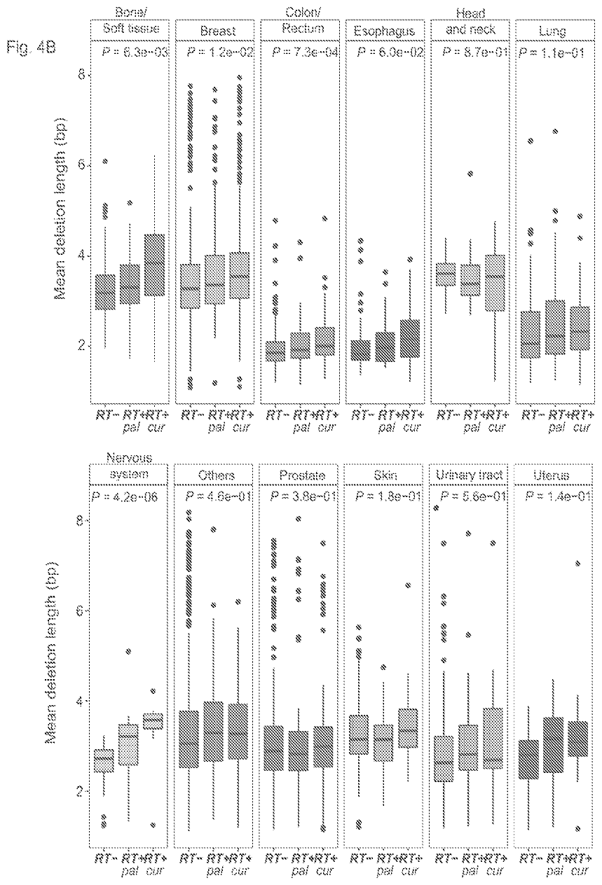

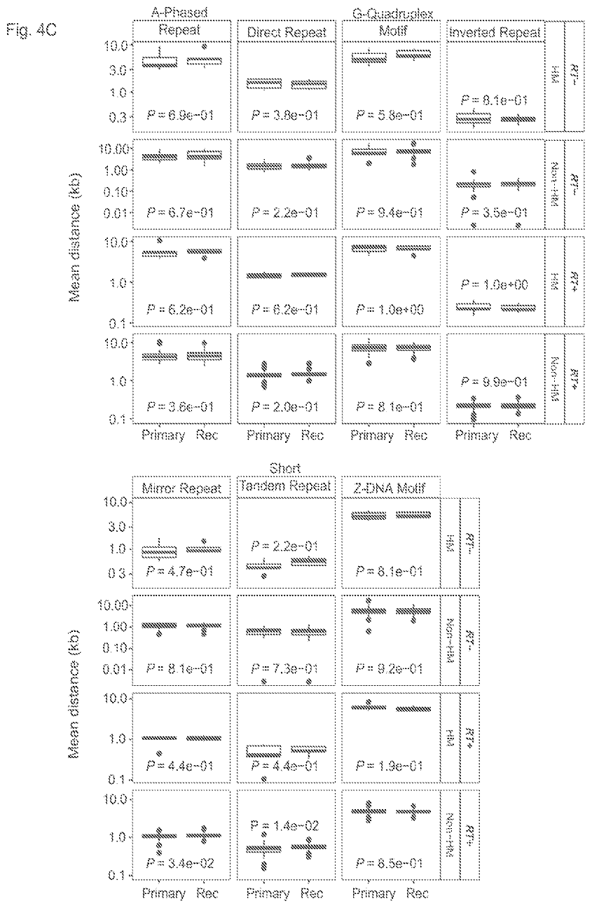

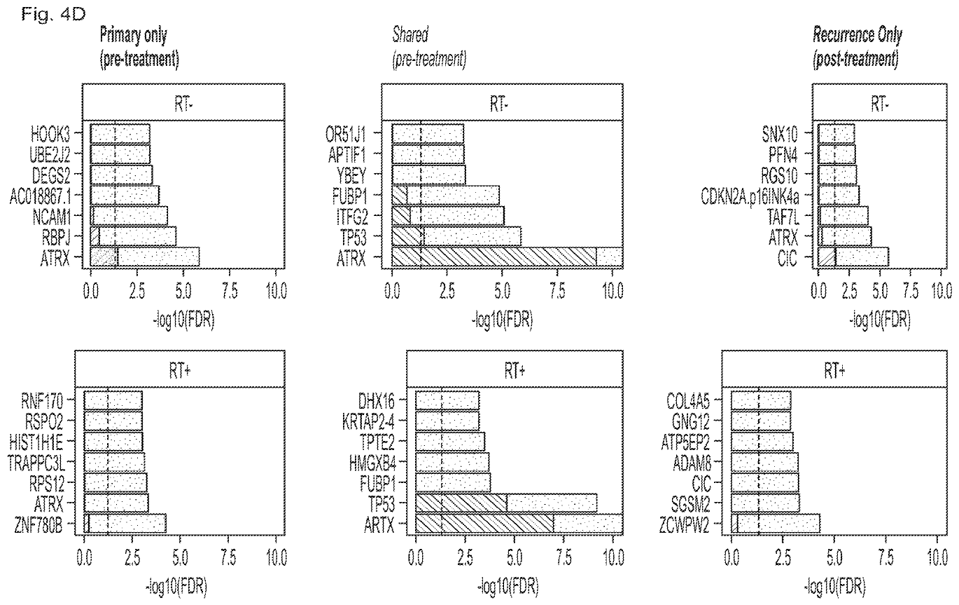

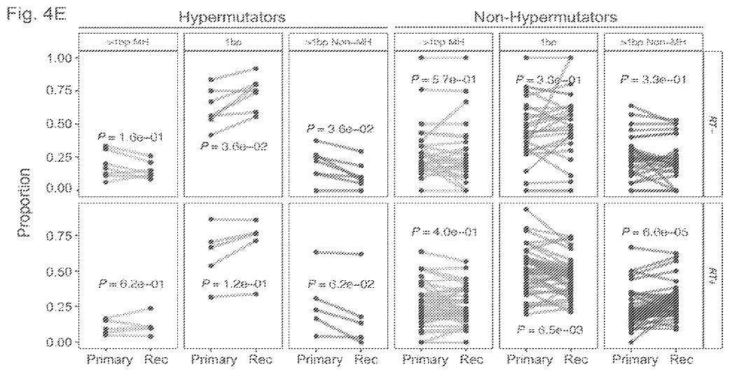

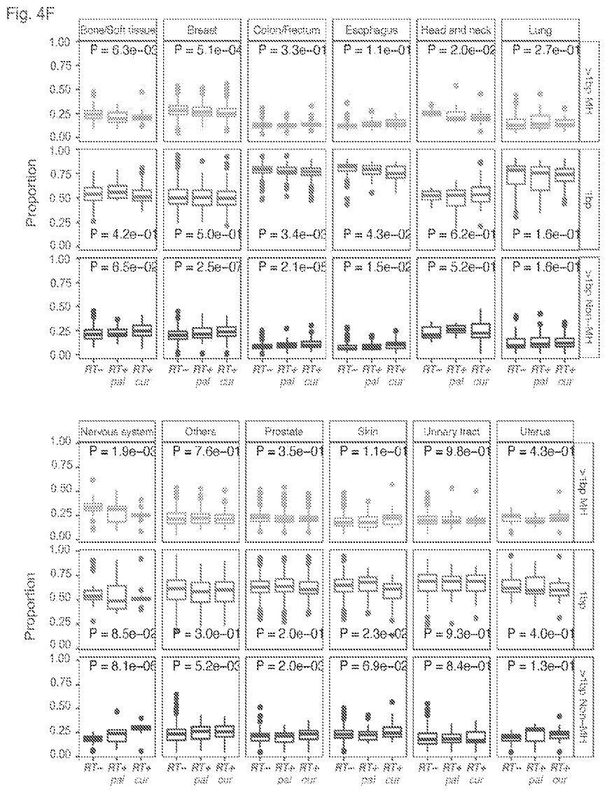

Characteristics of RT-associated small deletions, such as length distribution and breakpoint microhomology, may be able to provide insights on their etiology. Such features were explored in the GLASS dataset, limiting the analysis to IDH mutant gliomas (RT+, n=49; RT−, n=32) due to the imbalance in radiation treatment amongst IDH wild-type gliomas in the GLASS cohort (RT+, n=107 vs RT−, n=2). Small deletions in recurrent tumor samples that were treated with RT showed increased deletion lengths (FIG. 3A, upper panels, RT+: P=1.5e-04; RT−: P=3.5e-01, paired Wilcoxon signed-rank test). This increase was particularly associated with new deletions that occurred after therapy (FIG. 4A, P=1.3e-04, Mann-Whitney Utest), supporting the idea that RT leads to longer deletion lengths (FIG. 3A, upper panels). Moreover, a detailed analysis of the size distribution of deletions revealed a shift towards deletions of length ˜5-15 bp following RT-treatment in non-hypermutated gliomas (FIG. 3A, lower panel).

Comparing RT-treated and RT-naïve metastatic tumor samples from the single time-point HMF dataset showed a similar larger average deletion length for both palliative and curative RT-treated tumor samples (FIG. 3B, FIG. 4B). Moreover, a shift was also observed in deletion span from small 1-4 bp deletions towards medium-sized 5-15 bp deletions (FIG. 3B). A stepwise increase in deletion length was observed for palliative and curative RT-treatment, respectively, providing further evidence for a dose and exposure association. Taken together, these results indicated not only an increased deletion burden, but also highlighted distinct characteristics of RT-associated small deletions.

B-DNA is the common right-handed, double helical formation of DNA. Non-canonical non-B-DNA structures and fragile repeat-rich DNA may be more prone to acquiring mutations [Georgakopoulos-Soares, I. et al., Genome Res. 28, 1264-1271 (2018)]. Therefore, it was hypothesized that RT-induced deletions were more likely to occur in these fragile regions of the genome. The link between small deletions and these genomic features was investigated by adjusting for a random background distribution. Importantly, deletions following RT showed less variability and higher similarity to the random background distribution compared to non-RT-induced deletions (FIG. 3C, upper). Furthermore, comparison of GLASS pre- and post-treatment deletions indicated that deletions following radiotherapy showed larger distances to non-B DNA features (FIG. 3C, lower, FIG. 4C). The lack of or reduced association between radiation-induced deletions and the analyzed genomic features, such as repeats and G-quadruplex motifs, suggested that ionizing radiation associated small deletions occurred in a largely stochastic manner, occurring independently from the intrinsic mutagenicity of the fragile regions of the genome analyzed.

It was assessed whether RT-associated small deletions showed enrichment in driver genes. The covariate-adjusted normalized ratio between non-synonymous and synonymous mutations (dN/dS) was computed in order to identify selection of mutations at the level of individual genes separately for GLASS pre- and post-treatment fractions (FIG. 4D) [Martincorena, I. et al., Cell 171, 1029-1041.e21 (2017)]. Genes with dN/dS ratios strongly deviating from one were thought to be under selection and may be associated with the RT+ small deletion phenotype. No evidence was found for significant selection for any genes in the post-treatment fraction following radiation therapy. Because these analyses focused on acquired deletions only present after RT, they could not also be performed in the HMF set where pre-treatment samples were unavailable. The results in IDH-mutant glioma further supported the notion that RT-associated deletions did not occur at particular genomic loci, instead showing a more dispersed genomic spread.

Small deletions can be the result of error-prone DSB-repair utilizing mechanisms such as c-NHEJ and a-EJ/MMEJ [Chang, H. H. Y. et al, Nat. Rev. Mol. Cell Biol. 18, 495-506 (2017)]. To investigate whether a preference for DSB-repair pathway choice following RT existed, microhomology sequences were computed at breakpoints and deletions were characterized based on size, microhomology, and repeat content (FIG. 3D, FIG. 2E). Deletions without microhomology comprised the majority of deletions in the dataset (77%, FIG. 3D). However, in non-hypermutant gliomas receiving ionizing radiation a significant increase in >1 bp deletions without microhomology was observed (FIG. 3D, P=6.6e-05, Paired Wilcoxon signed rank test) and conversely a decrease in 1 bp-deletions was observed (FIG. 3D, P=6.5e-03, Paired Wilcoxon signed-rank test). Using the same three categories described in FIG. 3D, comparison of RT-treated and RT-naïve metastatic tumors from the HMF dataset demonstrated comparable results (FIG. 4F). These data suggested that c-NHEJ was the preferred pathway for repairing radiation-induced DNA damage.

### Example 3 Distinct ID and SBS Mutational Signatures Associated with Radiotherapy

### Materials and Methods

Materials and methods were as described in Examples 1 and 2 above and as below herein, as appropriate.

### Mutational Signatures

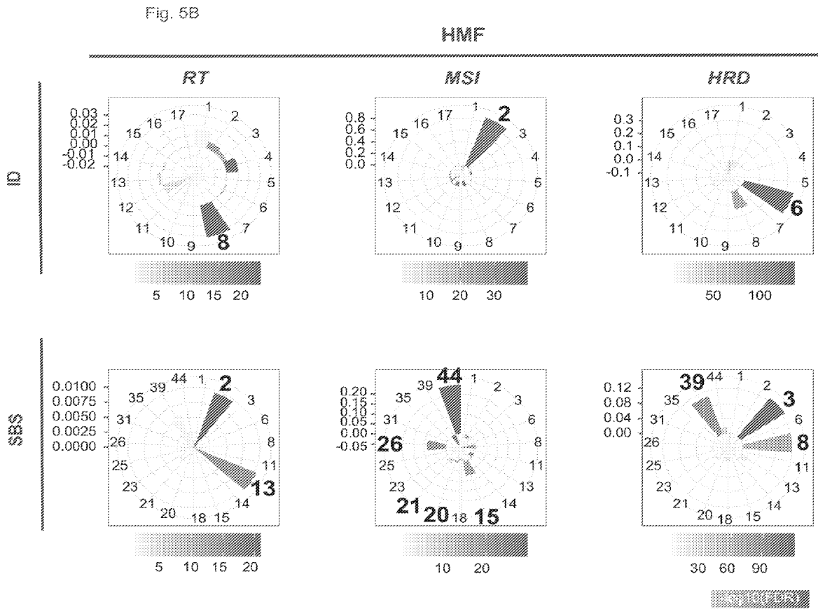

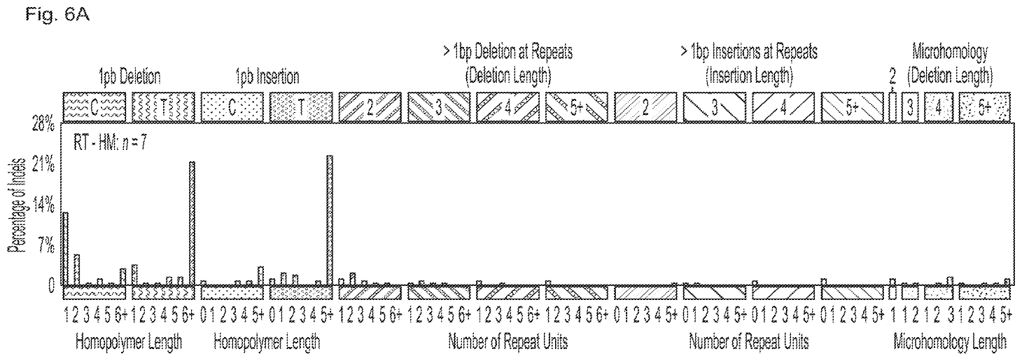

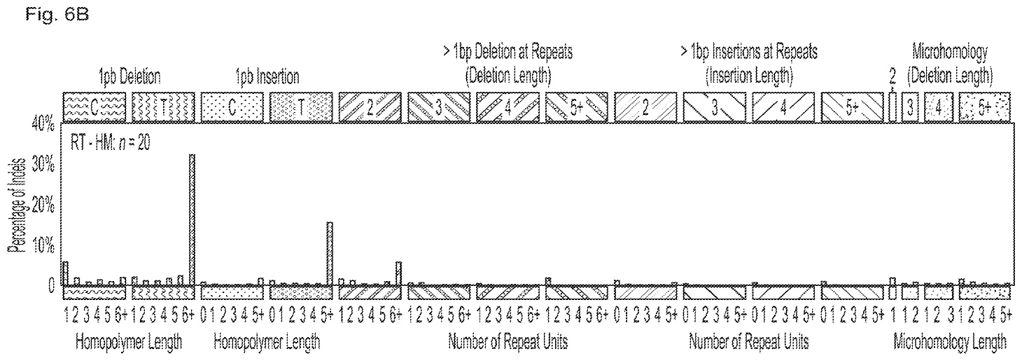

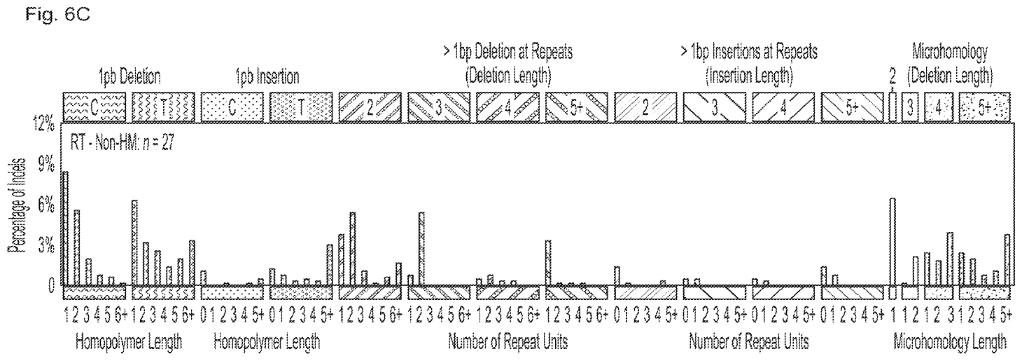

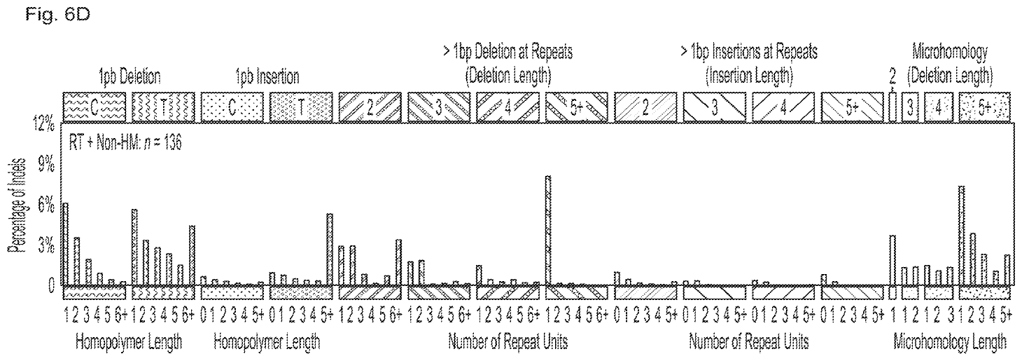

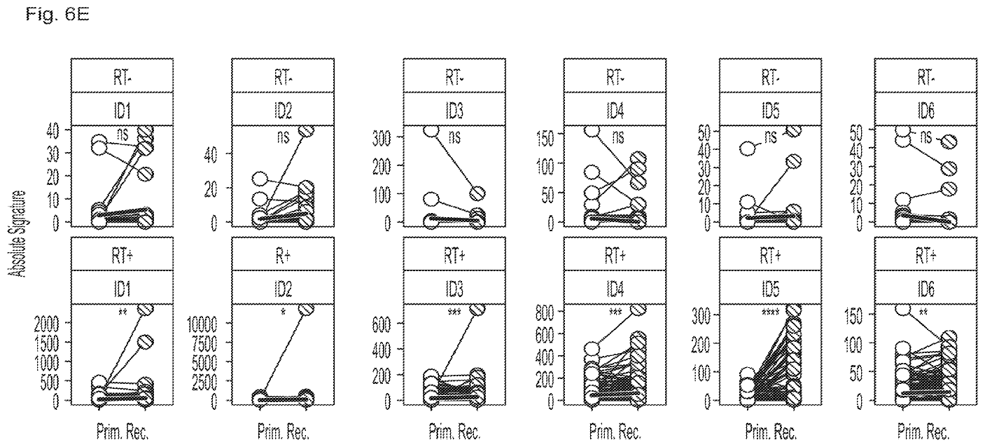

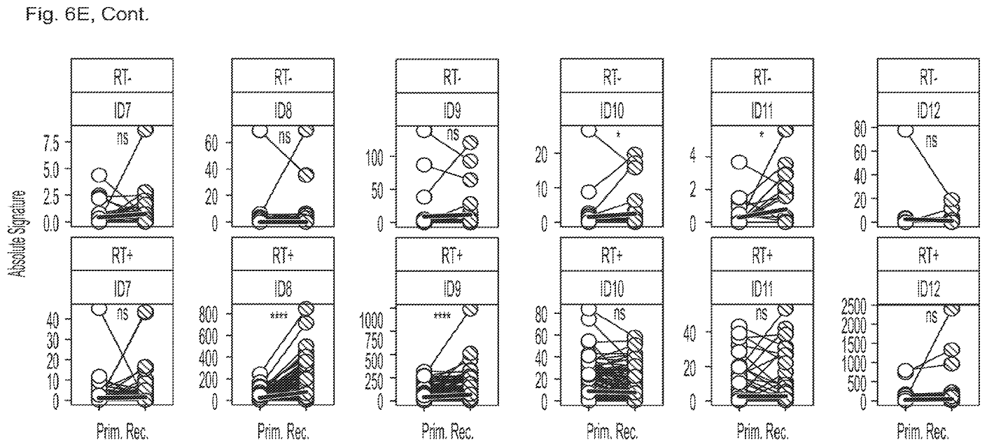

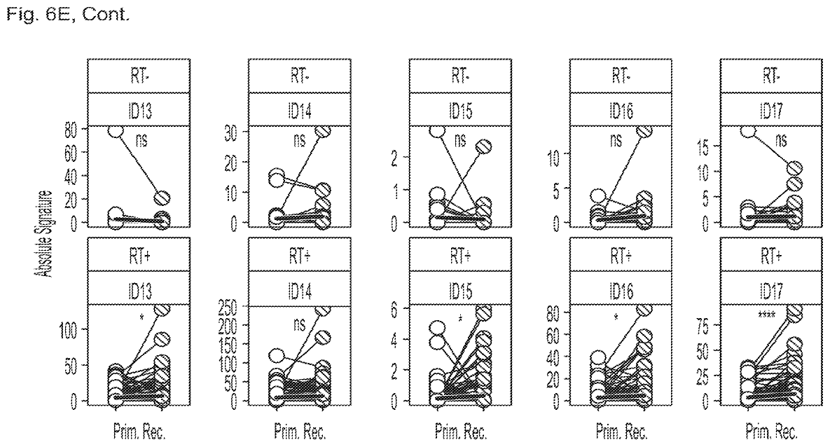

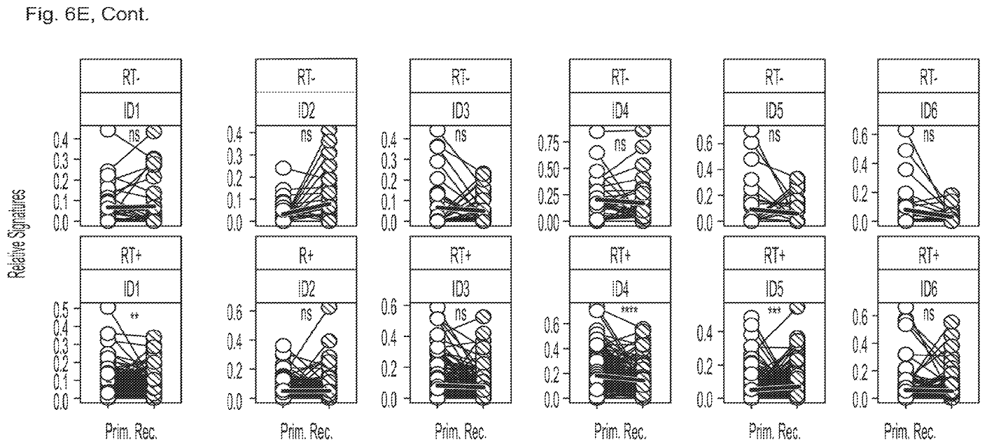

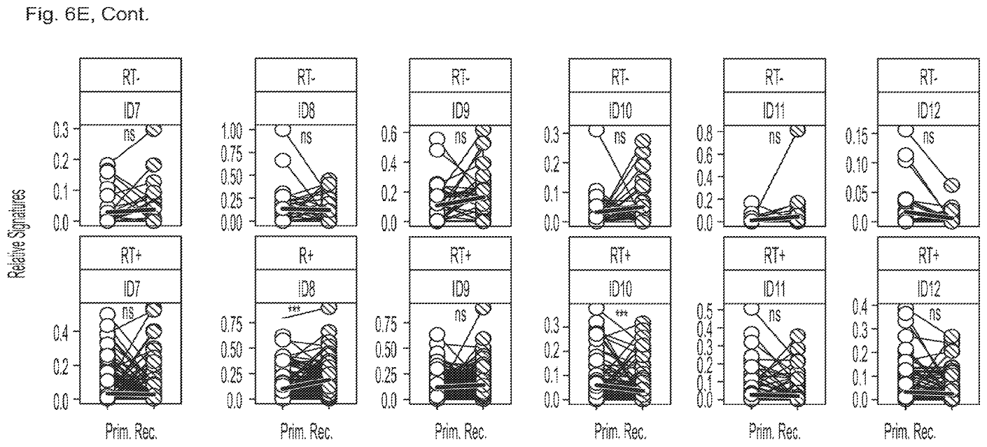

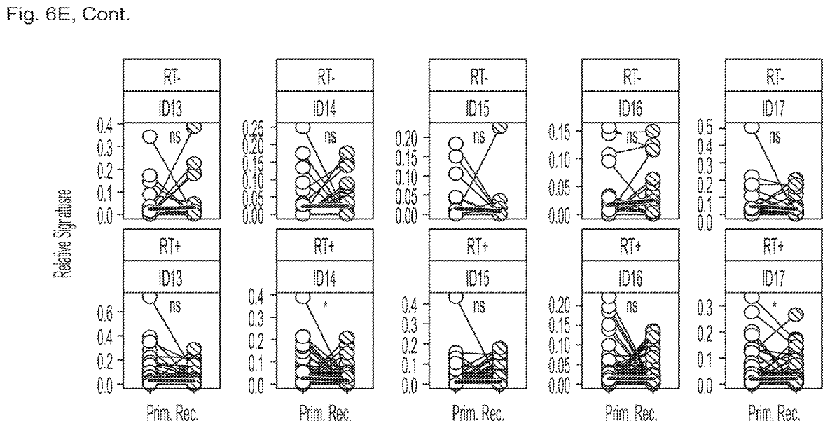

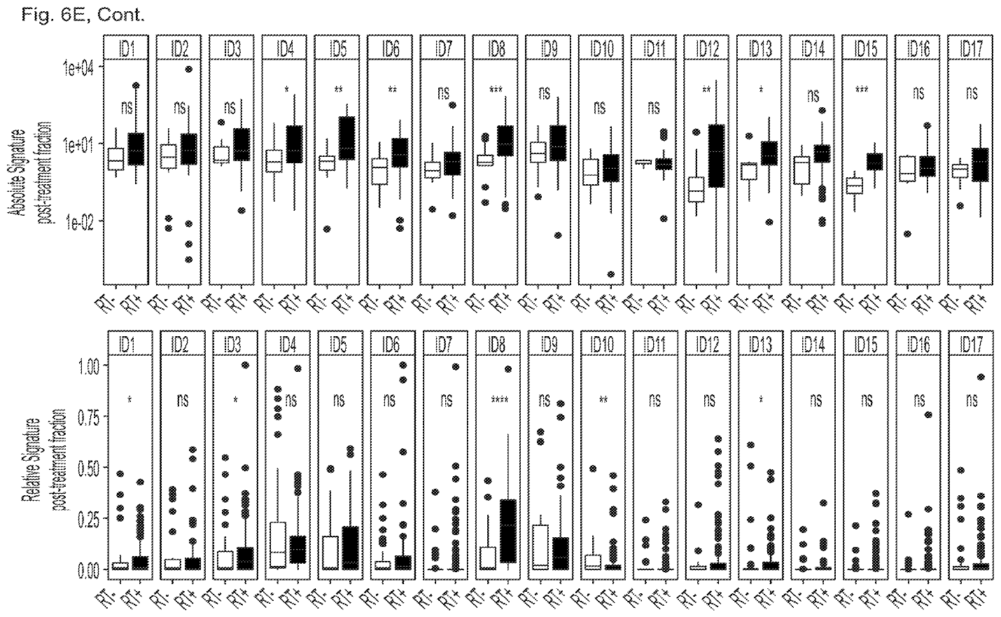

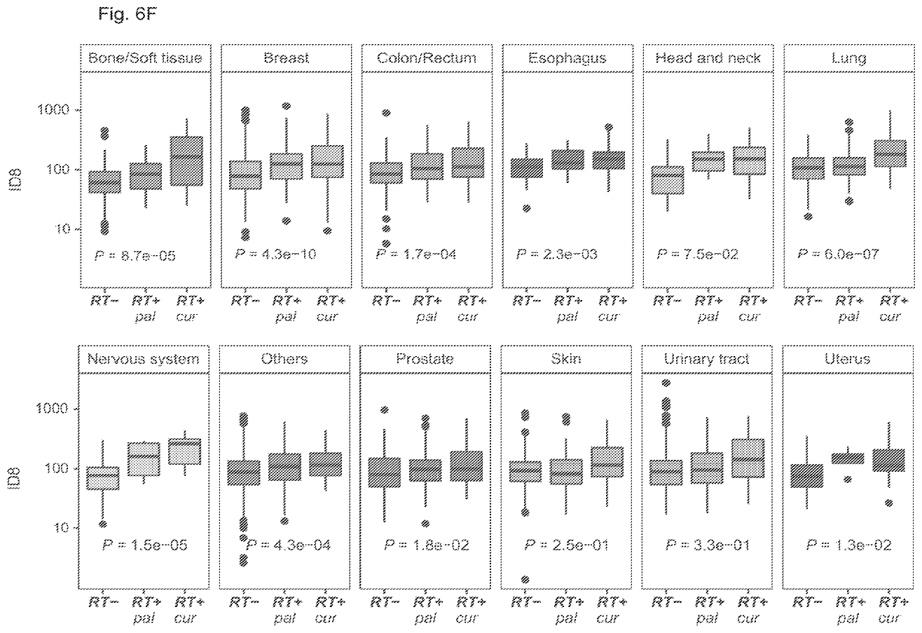

SigProfiler was used to extract and plot mutational signatures of single base substitutions (SBS), double base substitutions (DBS), and indels (ID) as previously described [Alexandrov, L. B. et al., Nature 578, 94-101 (2020)]. Absolute and relative contributions of signatures were determined using modified functions from the MutationalPatterns R package [Blokzijl, F., et al., Genome Med. 10, 33-33 (2018)]. Briefly, the mutational profile matrix generated with SigProfiler was fitted to the catalog of previously identified COSMIC mutational signatures (v3, May 2019) by solving the non-negative least squares problem. The single base substitution signatures SBS31 and SBS35 were previously linked to platinum therapy [Pich, O. et al., Nat. Genet. 51, 1732-1740 (2019); Alexandrov, L. B. et al., Nature 578, 94-101 (2020)]. Analysis of the HMF cohort using the extracted signatures confirmed these previously established associations, supported the identified signatures. SigProfilerPlotting [Bergstrom, E. N. et al., BMC Genomics 20, 685 (2019)] was used to visualize the distribution of indel characteristics (FIG. 6A-D).

### Results

Cancer cells accumulate somatic mutations that are caused by intrinsic and/or extrinsic mechanisms. The different mutational processes can leave distinct genomic scars, termed mutational signatures.

To validate the underlying mutational processes of radiotherapy, pre-and post-treatment mutations in the GLASS dataset were compared to previously defined mutational signatures [Alexandrov, L. B. et al., Nature 578, 94-101 (2020)]. The comparison of signature contributions between post-treatment mutations in RT-treated and RT-naïve IDH mutant glioma samples revealed a strong enrichment of indel signature 8 (ID8, FIG. 5, FIG. 6D, RT+, mean contribution=0.22, vs. RT−, mean contribution, P=7.4e-05, Q=3.8e-03, Mann-Whitney Utest and false discovery rate, respectively). Furthermore, in RT-treated patients but not RT-naïve patients comparing ID8 values before and after treatment resulted in significant increases in absolute (FIG. 6E, P=4.5e-07, Paired Wilcoxon rank-signed test) and relative (FIG. 6E, P=2.3e-03) ID8 contributions, respectively. Signature ID8 was composed of >5 bp deletions without microhomology and had previously been linked to DSB repair by c-NHEJ, providing further evidence that radiation-induced DSBs were primarily repaired via c-NHEJ [Alexandrov, L. B. et al., Nature 578, 94-101 (2020)]. Hypermutation in IDH mutant gliomas was associated with a significant enrichment of indel signature 2 (ID2, FIG. 5, FIG. 6A-B). ID2 comprised 1-bp deletions at homopolymers and had been reported previously to be elevated in DNA mismatch repair deficient cancers [Alexandrov, L. B. et al., Nature 578, 94-101 (2020)].

A previous analysis of mutational signatures in the HMF dataset found that of all indel signatures the strongest association with radiotherapy treatment was with COSMIC-ID6 (corresponding to SignatureAnalyzer-ID12) [Pich, O. et al., Nat. Genet. 51, 1732-1740 (2019)]. Consistent with findings in the GLASS cohort, it was observed that the strongest association was with ID8, and significant but substantially less pronounced for ID6 (FIG. 5). Both absolute and relative ID8 values were significantly higher in RT-treated samples when compared to RT-naïve samples, and a significant association was observed in nine of twelve tumor types (FIG. 6F). Importantly, the comparison of HRD+ and HRD− samples showed a clear association of HR-deficiency with ID6. ID6 comprised >5 bp deletions with microhomology at breakpoints and had previously been reported to be elevated in HR-defect breast cancers [Davies, H. et al., Nat. Med. 23, 517-525 (2017)]. Additionally, the findings from the GLASS cohort and previous observations that MSI samples were enriched for indel signature 2 (ID2, FIG. 5) were validated.

Collectively, these results have important implications for differential mutational processes acting on the cancer genome. While MSI leads to an increased burden in small deletions due to hypermutability resulting from impaired DNA mismatch repair at microsatellites/homopolymers, the DSBs induced by RT and due to HRD are repaired via error prone DSB-repair mechanisms. These results clearly suggested two different mechanisms for the repair of DSBs: the a-EJ pathway that utilizes microhomologies at breakpoints in HR-deficient samples (signature ID6) and the c-NHEJ pathway that does not require microhomologies at breakpoints in RT-treated samples (signature ID8).

Next, identification of single base substitution (SBS) signature associations in both datasets was investigated. Consistent with previous reports, significant enrichment of SBS11 was found in hypermutant IDH mutant glioma samples [Barthel, F. P. et al., Nature 576, 112-120 (2019); Touat, M. et al., (2020) Nature 580(7804), 517-523] and enrichment of signatures SBS44, SBS26, SBS21, SBS20, and SBS15 was found in MSI samples [Alexandrov, L. B. et al., Nature 578, 94-101 (2020)]. In HRD cases enrichment of SBS3 and SBS8 was observed, which was previously described [Alexandrov, L. B. et al., Nature 578, 94-101 (2020); Davies, H. et al., Nat. Med. 23, 517-525 (2017); Nik-Zainal, S. et al., Nature 534, 47-54 (2016)] along with a so far unreported enrichment of SBS39 (FIG. 5A-B).

### Example 4 Radiotherapy Treatment is Associated with Aneuploidy and Larger Deletions

### Materials and Methods

Materials and methods were as described in Examples 1-3 above herein, and as below herein, as appropriate.

### Structural Variants

For the GLASS dataset split reads and discordant read pairs were extracted from all tumor and normal BAM files using samtools 1.7 [Li, H. et al., Bioinformatics 25, 2078-9 (2009)]. The lumpyexpress tool (from LUMPY 0.2.13) was used to call structural variants providing the data associated with the set of normal and tumor samples belonging to one patient [Layer, R. M. et al., Genome Biol 15, R84 (2014)]. CNV predictions inferred from read-depth using CNVnator 0.3.3 were additionally provided to further support identified variants [Abyzov, A. et al., Genome Res. 21, 974-84 (2011)]. The resulting call set was post-processed using SVtyper 0.6.0 to genotype structural variants for each individual sample belonging to a patient [Chiang, C. et al., Nat. Methods 12, 966-8 (2015)]. Finally, GATK VariantFiltration was used to filter all variants with less than four reads of support and those with quality scores less than ten [Van der Auwera, G. A. et al., Curr. Protoc. Bioinformatics 11.10.1-11.10.33 (2013)]. Variants that showed any support in non-tumor samples were additionally removed. Variants were quantified per sample and further stratified according to type (translocation, duplication, deletion, and inversion). The change in frequencies for each patient was computed by dividing the rate at recurrence by the rate at primary. Only variants spanning at least 20 bp were considered.

### Aneuploidy Calculation

Arm-level aneuploidy data from the GLASS dataset was obtained from a previous publication and copy number segmentation files from HMF were processed into arm-level copy number calls as previously described [Barthel, F. P. et al., Nature 576, 112-120 (2019)]. Chromosomes demonstrating euploidy in both arms were considered euploid. Chromosomes with equidirectional aneuploidy in both arms or aneuploidy in a single arm and indeterminate ploidy in the other arm were considered “simple aneuploid”. Chromosomes with aneuploidy in one arm and incongruent ploidy in the other arm were considered “complex aneuploid”. Aneuploidy events were quantified for each tumor sample.

### Results

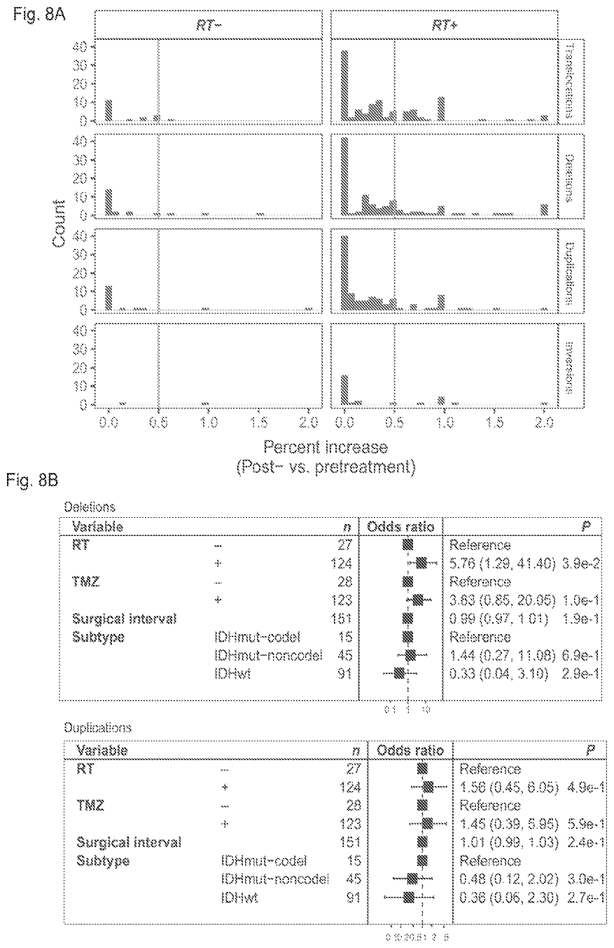

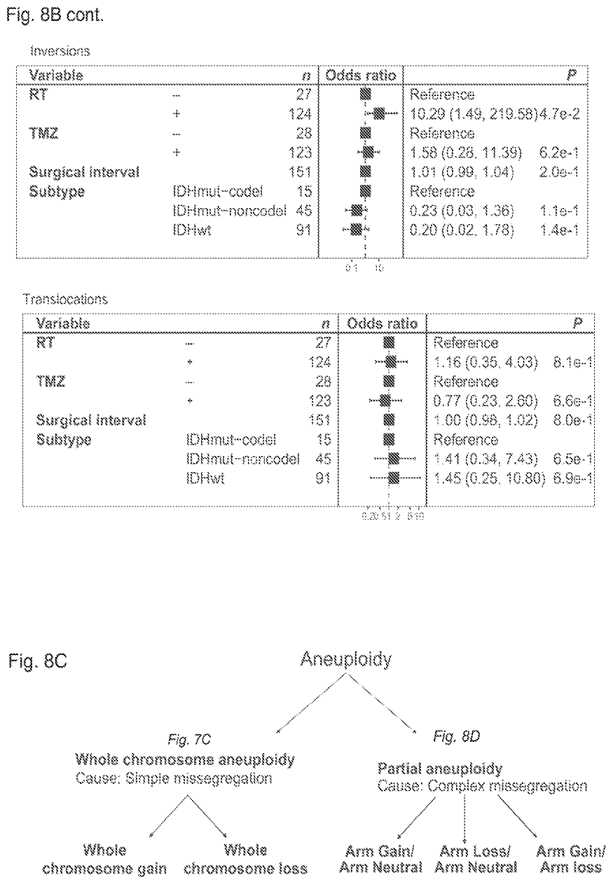

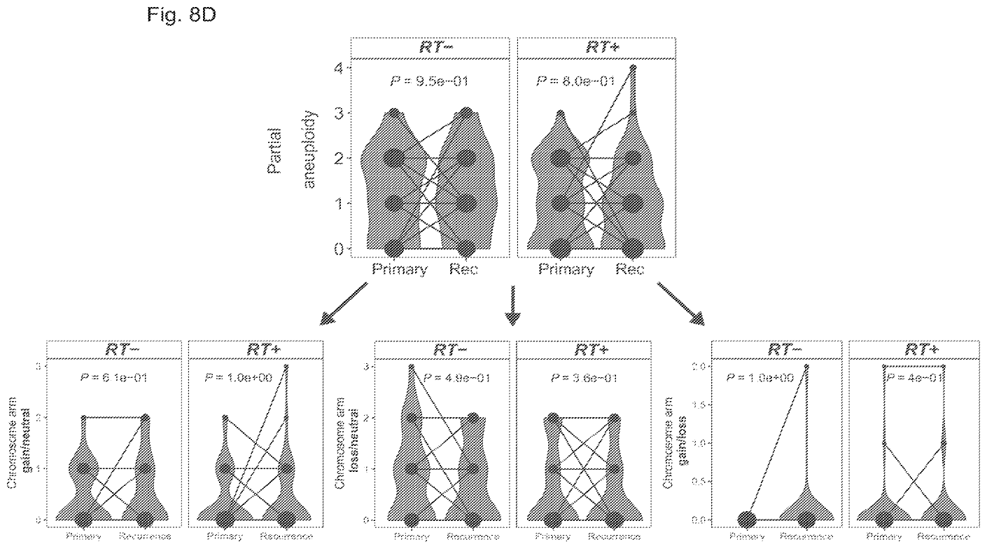

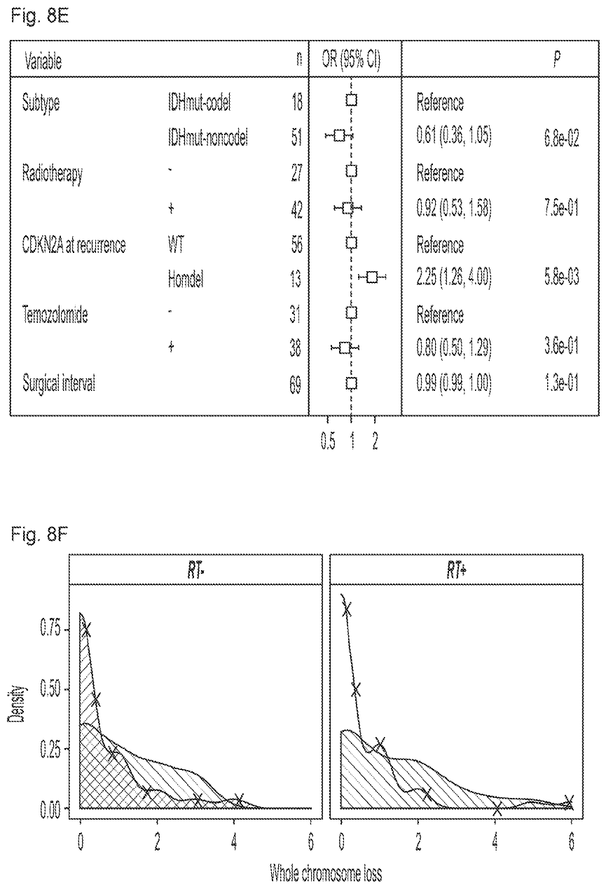

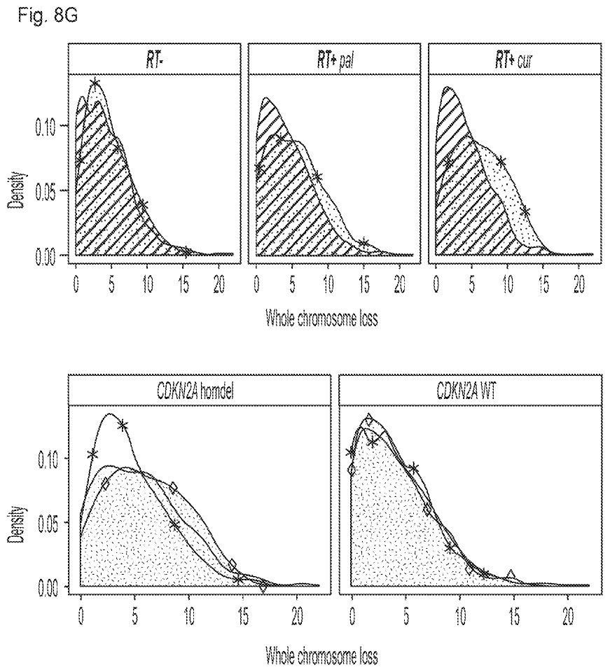

Having established an association with radiotherapy and small deletions as well as specific mutational signatures, it was reasoned that RT-induced DSBs may also result in other types of genomic damage. Large structural variants, including large deletions, duplications, inversions, and translocations, were detected in the longitudinal GLASS cohort, and it was observed that a statistically significant number of RT-treated patients demonstrated an increase of large deletions (length>20 bp to chromosome arm length) post-therapy that was not seen in RT-naïve patients (FIG. 7A, P=3.2 e-02, Fisher's exact test). Interestingly, a similar statistically significant increase in inversions was observed (FIG. 7A, P=2.1e-02), and no differences were observed for translocations (FIG. 7A, P=1) and duplications (FIG. 7A, P=7e-01). These associations remained significant after accounting for potentially confounding factors such as TMZ treatment and molecular subtype (FIG. 8B). While radiation-associated secondary malignancies have been reported to contain increased rates of inversions [Behjati, S. et al., Nat. Commun. 7, 12605 (2016)], a concomitant increase in large deletions in association with RT has not been previously observed.

The next evaluation was whether any specific deletions were associated with radiotherapy treatment. Comparing alteration frequencies before and after RT in the GLASS cohort identified a significant link between radiotherapy and gain of CDKN2A homozygous deletions among IDH-mutant gliomas where CDKN2A loss at diagnosis is rare [Ceccarelli et al., 2016, Cell 164, 550-563] (FIG. 5B). The IDH-wild type gliomas subgroup in the GLASS cohort lacked the number of RT− cases to perform the same analysis. CDKN2A homozygous deletions occurred exclusively in RT-treated recurrences (FIG. 7B) and occurred significantly more frequently than in pre-treatment samples (FIG. 7B, 29% vs. 2%, P=1.9e-05, Fisher's exact test), nominating acquired CDKN2A homozygous loss as a potential novel biomarker for RT-resistance among IDH-mutant gliomas, but not IDH-wild type gliomas where CDKN2A homozygous deletion at diagnosis is common.

Ionizing radiation can promote mitotic chromosome segregation errors causing aneuploidy [Adewoye, A. B., et al., Nat. Comm. 6, 6684-6684 (2015); Rose Li, Y., et al., Nat. Comm. 11, 394-394 (2020); Bakhoum, S. F., et al., Nat. Comm. 6, 5990-5990 (2015); Touil, N. et al., Mutagenesis 15, 1-7 (2000)]. Specifically, ionizing radiation can induce non-disjunction events during mitosis, leading to an imbalanced chromosomal copy number between two daughter cells [Behjati, S. et al., Nat. Commun. 7, 12605 (2016)]. The association of aneuploidy with radiation therapy was investigated, separating aneuploidy events into gains or losses of entire chromosomes, likely the result of segregation errors, and partial gains or losses, requiring additional DSBs (FIG. 8C). In an analysis of the IDH-mutant GLASS cohort, a significant association was observed between RT and chromosome losses, whereas no association was observed for simple gains or complex events (FIG. 7C, FIG. 8D). However, after adjusting for covariates in a multivariable Poisson regression model used to model integer counts of aneuploidy events, the effect of radiotherapy on chromosome losses in the GLASS cohort was no longer statistically significant. The analysis highlighted a significant association between chromosome losses and CDKN2A deletions (FIG. 8E), implicating that the increase in chromosome loss frequency following RT is specific to RT-associated acquired CDKN2A deletions. Using the Hartwig metastatic tumor cohort, an association between CDKN2A homozygous deletions and simple chromosome losses was demonstrated (FIG. 7D, FIG. 8F). Using Poisson regression to model associations of chromosome loss frequency showed that both curative RT-treatment and CDKN2A homozygous deletions were independently associated with increased number of chromosomal losses in the HMF dataset (FIG. 7D, FIG. 8F). However, testing for interactions between CDKN2A deletions and RT-treatment indicated a trend towards interaction between palliative/curative radiotherapy and CDKN2A deletions (Table 1, P=9.75e-02 and P=4.92e-02, respectively, t-test). As shown in Table 1, CDKN2A homdel and curative RT were independently associated with higher whole chromosome losses. These results suggest that chromosome segregation may not directly be associate with radiotherapy but through interactions with CDKN2A deletions. Thus, whereas radiotherapy may promote mis-segregation alone, when combined with CDKN2A homozygous deletions, this further exacerbates segregation errors and may generate additional aneuploidy.

TABLE 1
Multivariable Poisson regression model for whole chromosome losses
in metastatic cohort including tumor type, RT, CDKN2A status,
and an interaction term between RT and CDKN2A as variables.

| | | | Odds Ratio | |
|---|---|---|---|---|
| Variable | Levels | n | (95% CI) | P-value |
| Tumor type | Bone/Soft tissue | 179 | (ref) | |
| | Breast | 716 | 1.10 (1.01-1.20) | 2.50E−02 |
| | Colon/Rectum | 445 | 1.42 (1.30-1.54) | 1.11E−15 |
| | Esophagus | 128 | 1.89 (1.71-2.08) | <2E−16 |
| | Head and Neck | 53 | 1.05 (0.90-1.22) | 5.12E−01 |
| | Lung | 355 | 1.73 (1.59-1.88) | <2E−16 |
| | Nervous System | 74 | 0.51 (0.43-0.60) | 2.45E−14 |
| | Others | 810 | 1.16 (1.07-1.26) | 2.90E−04 |
| | Prostate | 392 | 0.53 (0.48-0.59) | <2E−16 |
| | Skin | 324 | 0.86 (0.78-0.94) | 1.50E−03 |
| | Urinary Tract | 163 | 1.05 (0.94-1.17) | 3.87E−01 |
| | Uterus | 54 | 1.03 (0.88-1.20) | 7.37E−01 |
| Radiotherapy | RT− | 2265 | (ref) | |
| | RT + pal | 689 | 1.04 (0.99-1.08) | 1.30E−01 |
| | RT + cur | 739 | 1.05 (1.01-1.10) | 2.40E−02 |
| CDKN2A | WT | 3065 | (ref) | |
| status | Homdel | 628 | 1.20 (1.14-1.26) | 1.79E−12 |
| Interaction | RT + pal: | | | 9.75E−02 |
| terms | CDKN2A homdel | | | |
| | RT + cur: | | | 6.92E−02 |
| | CDKN2A homdel | | | |

### Example 5 RT-Driven Genomic Changes Result in Poor Survival

### Materials and Methods

Materials and methods were as described in Examples 1-4 above herein, as appropriate.

### Results

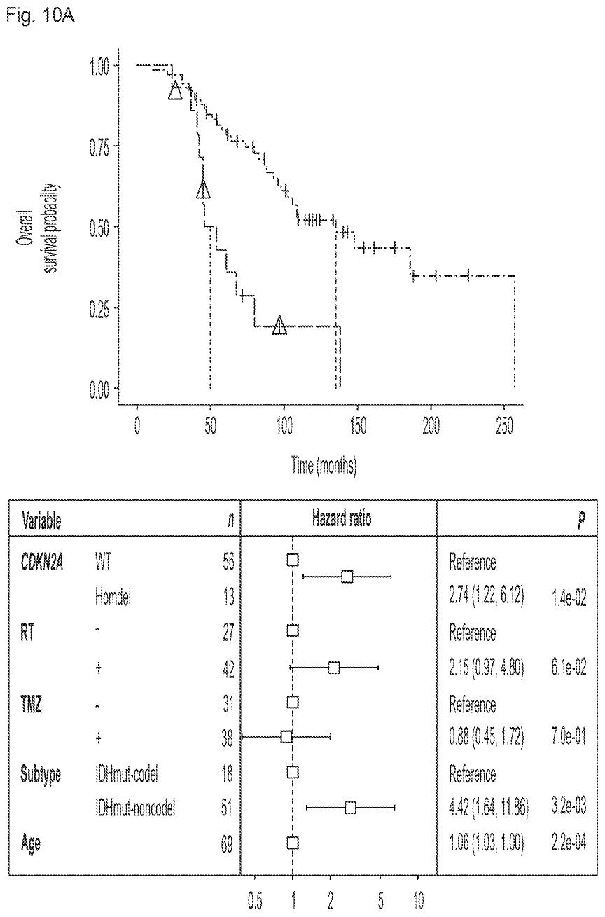

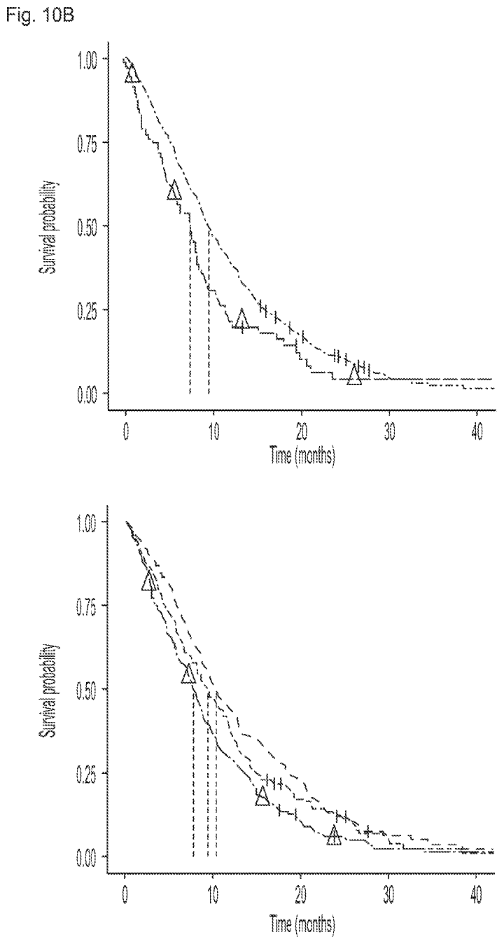

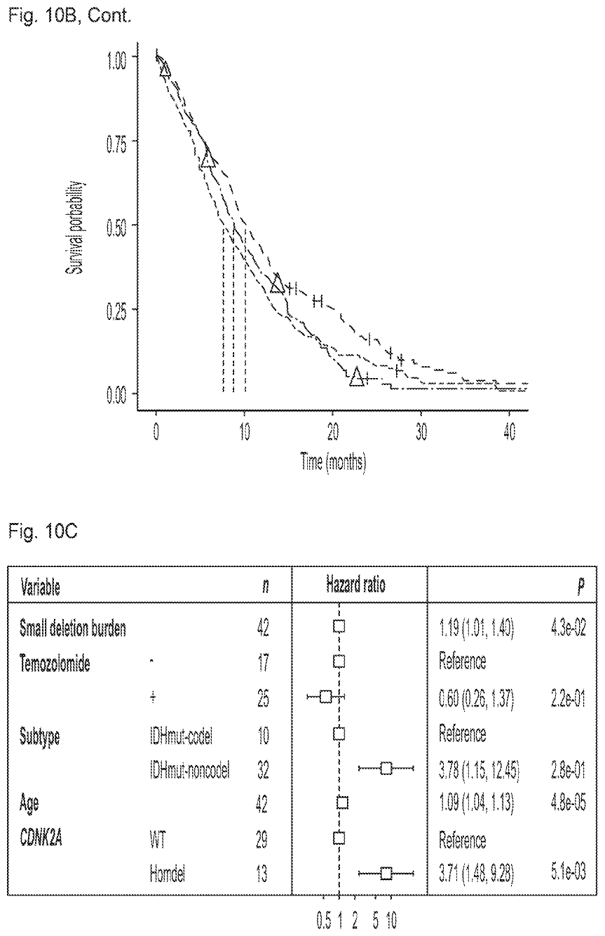

The final goal was to ascertain whether the genomic effects of radiotherapy were relevant to patient outcome. Survival analysis confirmed that CDKN2A homozygous deletion at recurrence was significantly associated with worse overall survival in IDH-mutant glioma samples (FIG. 10A, upper, P<1e-04, log-rank test), independent of age, treatment, or subtype (FIG. 10A, lower, P=1.4e-02, Wald test) [Barthel, F. P. et al., Nature 576, 112-120 (2019)]. To test for a survival association of CDKN2A deletions amongst RT-treated patients in the HMF dataset, 958 samples that received RT and had sufficient survival information available were selected from 11 tumor types (FIG. 2E). Although CDKN2A homozygous deletions are less common outside glioma and HMF data lacks longitudinal samples to limit the analysis to cases with acquired CDKN2A deletion, patients whose tumors harbored a CDKN2A homozygous deletions showed worse outcomes compared to patients with CDKN2A wild-type tumors (FIG. 10B, first panel). Stratification of the cohort into tertiles based on genome-wide aneuploidy frequency demonstrated that low aneuploidy was linked to favorable outcomes and high aneuploidy was linked to poor outcomes (FIG. 10B, second panel). These results indicated that acquired CDKN2A homozygous deletion was a biomarker of RT resistance after recurrence and supported the clinical reassessment of CDKN2A status at recurrence for optimizing treatment strategies, particularly not only as single time point as previously reported [Appay, R., et al., Neuro-oncology 21, 1519-1528 (2019); Shirahata, M., et al., Acta Neuropathologica 136, 153-166 (2018); van Thuijl, H. F., et al., Genome Biology 15, 471-471 (2014)]—but also longitudinal prognostic markers in IDH-mut glioma.

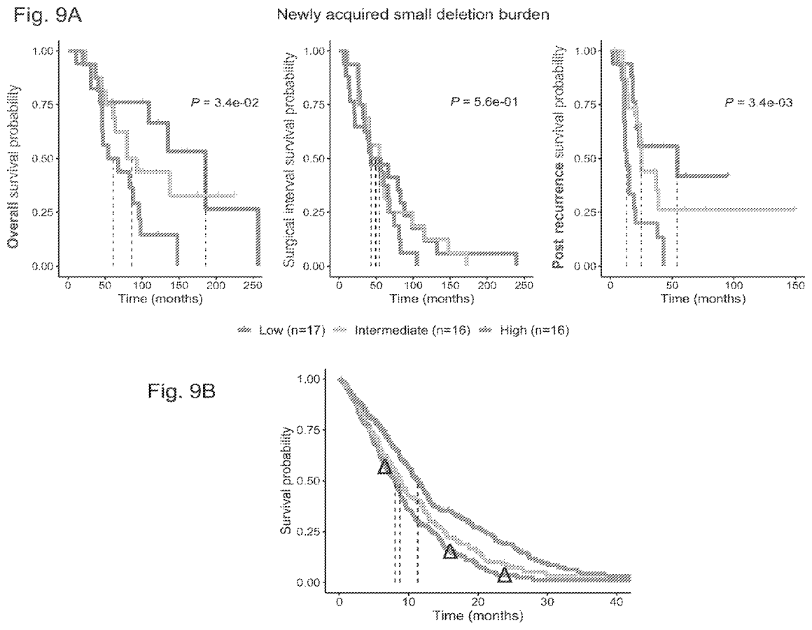

Independent of the poor prognostic implications of specific, RT-induced changes such as CDKN2A deletions, it was found that GLASS patients with tumors carrying a high small deletion frequency at recurrence (top tertile) had significantly shorter overall survival (FIG. 9A, P=3.4e-02, log-rank test). The association remained significant when accounting for the small deletion burden as a continuous variable and possible confounding variables, indicating a robust correlation (FIG. 10C, HR=1.19 [95% CI. 1.01-1.14]; P=4.3e-02, Wald test). Multivariable modeling using a limited subset of patients with detailed dosage information in the GLASS cohort (n=21) further indicated that the association with the small deletion burden and survival was independent of dose, P=2e-02). Separating the overall survival time into surgical interval and post-recurrence survival indicated that the association of high newly acquired small deletion burden with worse survival was limited to post-recurrence survival (FIG. 9A, P=3.4e-03, log-rank test). Surgical interval times did not differ significantly between the three tertiles (FIG. 9A, P=5.6e-01), suggesting that glioma patients may initially benefit equally from RT, but after certain exposure to RT and acquisition of the deletion signature, patients may lose sensitivity to further radiotherapy.

The association between radiotherapy and patient outcome in the HMF metastatic tumor datasets was then determined using the 958 RT-treated patients described above herein (FIG. 2G). Using this cohort, patients harboring a high small deletion burden (top tertile) were found to have significantly shorter survival than other RT-treated cases (FIG. 9B, P<4e-04, log-rank test). Similarly, HMF patients could be stratified into tertiles according to ID8 burden to show that an intermediate or high ID8 burden was associated with poor survival and a low ID8 burden was associated with improved outcomes (FIG. 10B, third graph). The effect of RT-associated small deletion burden on the survival of the patients was further independent of mutations in DDR genes. This validation of a worse outcome association in a single-time point analysis suggested that the presence of a higher number of RT-associated small deletions implicated a tumor that had responded to therapy, but which may have lost some or all of the treatment sensitivity. Taken together, these results support a finding that a higher deletion burden reflects a scenario that is favorable to a tumor, characterized by proficient DNA repair resulting in less tumor cell killing and decreased treatment efficacy, and that a higher deletion burden as a result of radiation therapy reflects a more aggressive tumor with increased levels of resistance to follow up treatments.

### Discussion

The studies described above herein in Examples 1-5 comprehensively evaluated the effects of ionizing radiation on the cancer genome using a cohort of pre- and post-treatment glioma samples and a cohort of metastatic tumor specimens. A unique signature of RT-associated deletions was identified. It was found that CDKN2A homozygous deletions were acquired in RT-treated IDH-mutant gliomas but not in untreated recurrent IDH-mutant gliomas, supporting a conclusion that radiotherapy-induced DNA damage promoted the acquisition of this poor prognostic marker. Further, it was found that a higher load of RT-induced deletions associated with worse patient outcomes, supporting a conclusion that the increased deletion burden reflected the repair of RT-induced DNA damage.

The genomic impact of therapeutic radiation has not been previously comprehensively shown, implicating a knowledge gap. Radiotherapy is used in the clinical management in over 50% of cancer patients [Barton, M. B. et al. Radiother. Oncol. 112, 140-4 (2014); Tyldesley, S. et al., Int. J. Radiat. Oncol. Biol. Phys. 79, 1507-15 (2011)] and effectively the most widely used regiment for cancer treatment. Prior studies on radiation induced tumors have shown a wide range of genomic effects and have suggested the involvement of various DNA double strand break repair mechanisms [Rose Li, Y. et al., Nat. Commun. 11, 394 (2020); Behjati, S. et al., Nat. Commun. 7, 12605 (2016); Davidson, P. R. et al., Sci. Rep. 7, 7645 (2017); Lopez, G. Y. et al., Acta Neuropathol. 137, 139-150 (2019); Phi, J. H. et al., Acta Neuropathol. 135, 939-953 (2018)]. The findings herein that RT was associated with a significantly higher burden of small deletions harboring specific genomic signatures, large deletions, large inversion and whole chromosome loss-driven aneuploidy extends the knowledge base and provides direction for development of effective radiosensitizers.

This work supports a conclusion that these events are a consequence of RT-induced mutagenesis/repair cycles. While the nearly exclusive association between acquired CDKN2A deletions and RT-treatment supports a conclusion that these events are RT-induced. The significant expansion of clones harboring RT-induced genomic events depends on clonal selection or drift [Reiter, J. G. et al., Nat. Genet. 52, 692-700 (2020)]. Therefore, the increased small deletion burden in combination with poor outcomes appeared to reflect the emergence of more competitive clones under RT-induced stress, innately active repair processes ensuring tumor maintenance, or a combination of these two. Thus, additional rounds of RT in patients with recurrent or metastatic tumors containing a significant increase in small deletion burden is unlikely to further extend progression-free survival. The assessment of small deletions may be used as a means with which to readily detect increased small deletion burden may help reduce costs of treatment and avoid RT-associated patient comorbidities and side-effects.

Example 6 Detecting a ‘small deletion phenotype’ from circulating tumor DNA and determining whether additional radiation treatment could be effective Circulating tumor DNA (ctDNA) is isolated from blood or cerebrospinal fluid using commercially available methods such as from Qiagen (Qiagen, Germantown, MD), and may be used to identify tumor-type-specific signatures [Nassiri et al., Nat. Med. 26, 1044-1047 (2020)]. The burden of small deletions, a normalized approximation of the total number of small deletions across the genome, is compared to the burden of small deletions detected in the genome of a tumor specimen. When the positive difference between ctDNA-derived and tumor specimen-derived small deletion burden exceeds a certain threshold, which is either an absolute (+0.5 small deletions/Mb) or a relative increase (50% additional new small deletions), it may be decided that additional treatment regimens based on ionizing radiation will not be effective. Thus, information obtained from assessing the burden of small deletions in a subject is used to select a treatment regimen for the subject, and the subject is administered the selected treatment regimen.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated herein in their entirety herein by reference.

What is claimed is:

1. A method of selecting a cancer treatment for a subject, comprising:
(a) obtaining a biological sample from a subject;
(b) determining at least one characteristic of a plurality of small deletions in somatic DNA in the biological sample;
(c) comparing the determined small deletions characteristic to a control of the small deletion characteristic previously determined for the subject; and
(d) selecting a treatment for the subject based at least in part on the comparison of the determined characteristic of the small deletions with the control.

2-3. (canceled)

4. The method of claim 1, wherein the at least one characteristic is one or more of: presence of the plurality of small deletions, small deletion burden, absence of the plurality of small deletions, absolute quantity of the small deletions in the plurality of small deletions, relative quantity of the small deletions in the plurality of small deletions, size of the small deletions in the plurality of small deletions, and genetic identity of one or more of the plurality of the small deletions.

5. The method of claim 1, wherein a means for determining the at least one characteristic of the plurality of small deletions comprises one or more of exome sequencing and whole genome sequencing.

6. The method of claim 1, wherein the subject has been administered a radiotherapy before the biological sample is obtained.

7. The method of claim 6, wherein the radiotherapy was administered to treat the cancer in the subject.

8. The method of claim 1, wherein the cancer is a metastatic cancer, or a recurrence of a previous cancer.

9. (canceled)

10. The method of claim 7, wherein the control of the small deletion characteristics comprises the small deletion characteristics previously determined for the subject before the radiotherapy was administered.

11. The method of claim 1, wherein the characteristic of the plurality of small deletions is a number of the small deletions and if:
(a) the number of small deletions determined in the subject sample is higher than a control number of small deletions, the selected treatment comprises abstaining from administering a radiotherapy to the subject, and
(b) the number of small deletions determined in the subject sample is statistically significantly equal to or lower than a control number of small deletions, the selected treatment comprises administering a radiotherapy to the subject.

12. The method of claim 11, wherein the determined number of small deletions in the subject sample is at least 10% higher than the control number of small deletions.

13. The method of claim 11, wherein the determined number of small deletions in the subject sample is at least 0.25, 0.5, 1, 2, 3, 4, 5, 6, 7, 8, 9, 10-fold higher than the control number of small deletions.

14. The method of claim 11, wherein if the control number of small deletions is zero and the determination of a number of small deletions in the subject sample is statistically significantly greater than zero, the selected treatment comprises abstaining from administering a radiotherapy to the subject.

15. The method of claim 1, wherein the characteristic of the plurality of small deletions is small deletion burden and if:
(a) the small deletion burden determined in the subject sample is statistically significantly higher than a control small deletion burden, the selected treatment comprises abstaining from administering a radiotherapy to the subject, and
(b) the small deletion burden determined in the subject sample is statistically significantly lower or is equal to a control small deletion burden, the selected treatment comprises administering a radiotherapy to the subject,
wherein a means for determining the small deletion burden comprises determining the at least one characteristic of the plurality of small deletions in somatic DNA in the biological sample.

16. (canceled)

17. The method of claim 15, wherein the determined small deletion burden in the subject sample is at least 10% higher than the control small deletion burden.

18. The method of claim 15, wherein the determined small deletion burden in the subject sample is at least 0.25, 0.5, 1, 2, 3, 4, 5, 6, 7, 8, 9, 10-fold higher than the control small deletion burden.

19. The method of claim 15, wherein if the control small deletion burden is zero and the determination of a small deletion burden is statistically significantly greater than zero in the subject sample, the selected treatment comprises abstaining from administering a radiotherapy to the subject.

20. The method of claim 1, wherein the determining of the characteristic of the plurality of small deletions comprises determining the identity of one or more specific small deletions in the plurality of small deletions.

21. The method of claim 1, wherein a selected treatment further comprises one or more of: surgery, chemotherapy, administration of a pharmaceutical agent, an immunotherapy, a modified T cell therapy, a virus-based therapy, abstention from surgery, abstention from chemotherapy, abstention from radiotherapy, radiotherapy, and abstention from administration of a pharmaceutical agent.

22. The method of claim 1, wherein the biological sample comprises one or more of: tissue, blood, serum, saliva, lymph fluid, circulating tumor DNA (ctDNA), cells of the subject, cancer cells of the subject, and cerebrospinal fluid (CSF).

23-25. (canceled)

26. The method of claim 1, wherein the cancer comprises: a brain cancer, a glioma, a bone cancer, a soft-tissue cancer, a breast cancer, a colon cancer, a rectal cancer, an esophageal cancer, a head and neck cancer, a lung cancer, an ovarian cancer, a prostate cancer, a skin cancer, a urinary tract cancer, or a uterine cancer.

27-29. (canceled)

30. The method of claim 1, wherein the at least one characteristic of the plurality of small deletions is an amount of the small deletions and the amount is at least 0.5 small deletions per megabase of sequence.

31. The method of claim 1, wherein the at least one characteristic of the plurality of small deletions is length of the small deletions in the plurality of small deletions and the small deletions are between 5 and 100 base pairs (bp) in length.

32. The method of claim 1, wherein the at least one characteristic of the plurality of small deletions is length of the small deletions in the plurality of small deletions and the small deletions are between 5 and 50 bp in length.

33. (canceled)

34. The method of claim 1, wherein the at least one characteristic of the plurality of small deletions is length of the small deletions in the plurality of small deletions and the small deletions are less than 100 bp in length.

35. The method of claim 1, wherein the at least one characteristic of the plurality of small deletions is length of the small deletions in the plurality of small deletions and the small deletions are of two or more different lengths.

36-75. (canceled)
